(19) United States
(12) Patent Application Publication
(10) Pub. No.: US 2015/0073043 A1
(43) Pub. Date: Mar. 12, 2015

(54) USE OF PHOSPHORYLATION PATHWAY-RELATED FACTOR IN REGULATING FUNCTION OF REGULATORY T CELL

(71) Applicant: Institut Pasteur of Shanghai, Chinese Academy of Sciences, Shanghai (CN)

(72) Inventors: Bin Li, Shanghai (CN); Zhiyuan Li, Shanghai (CN); Fang Lin, Shanghai (CN); Zhimei Gao, Shanghai (CN); Zuojia Chen, Shanghai (CN); Andy Tsun, Shanghai (CN); Kongchen Li, Shanghai (CN)

(21) Appl. No.: 14/373,107

(22) PCT Filed: Jan. 21, 2013
(86) PCT No.: PCT/CN2013/070769

§ 371 Date: Nov. 7, 2014

(30) Foreign Application Priority Data
Jan. 19, 2012 (CN) 201210017885.7

(51) Int. Cl. G01N 33/68; A61K 31/352

(52) U.S. Cl. CPC G01N 33/6872; A61K 31/352; G01N 2500/10
USPC 514/456; 435/375; 435/7.1

(57) ABSTRACT

A method for the treatment and/or the prevention of a disease or a symptom related to dysfunction of regulatory T cell immunomodulation includes administering to a subject in need thereof compositions that regulate regulatory T cell immunomodulatory function, in which the compositions may be prepared by contacting starting materials with phosphorylation pathway-related factors, the agonists or the antagonists thereof. The phosphorylation pathway-related factors are selected from: proto-oncogene protein PIM1 and the coding sequence thereof. The regulation is achieved by regulating the activity of regulators of regulatory T cells selected from the group: FOXP3, IL-2, GITR, CTLA4, and a combination thereof.

### BACKGROUND OF INVENTION

1. Field of the Invention

The present invention relates to molecular biology and biomedical fields. More specifically, the present invention relates to use of phosphorylation pathway-related factors, agonists thereof, or antagonists thereof to regulate regulatory T cell function through regulating the activities of FOXP3, IL-2, GITR, and CTLA4. The present invention especially relates to negative regulators that regulate FOXP3 activity and their immunosuppressive function produced by regulating regulatory T cells.

2. Background Art

FOX family of transcription factors is a large family of transcription factors having characteristics of forkhead/winged helix (FKH) and different functions. These family members play different roles in each process of cell development. (See References 1 and 2).

FOXP3 is a specific transcription factor of regulatory T cells (Treg). It plays an important role in regulating Treg development and function and has a function of down-regulating immune response. The FOXP3-mediated immune regulatory function of Treg is through forming FOXP3 protein complexes with certain transcription co-regulatory proteins (e.g., transcription factors, co-suppressors, co-activators, histone proteins, and chromatin remodeling factors) to dynamically regulate specific gene transcription. (See References 3 and 4). Under different stimulatory conditions, different states of FOXP3 protein complexes and their transcriptional activities have different effects on Treg and immune system regulation. (See References 3 and 4).

FOXP3+ Tregs belong to a T cell subset (i.e., CD4+ CD25+ Treg) in T lymphocytes expressing CD4, CD25, and FOXP3 transcription factor. Their normal functions are essential for dynamic regulation of body immune homeostasis. Dysregulation of regulatory T cell development and function is closely associated with physiological and pathological processes of a variety of major immune-related diseases, including autoimmune diseases, inflammation, acute and chronic infectious diseases, tumor immune tolerance, graft rejection, and allergic diseases. (See References 1 and 2).

Despite rapid progress in this field of research in recent years, many major and fundamental problems, however, still remain to be solved. For example, how regulatory T cells, on cellular level, regulate the activities of other immune cells, and what mechanisms, on molecular level, FOXP3 uses to cause regulatory T cells acquire immunosuppressive activity.

Accumulating experimental data show FOXP3 gene expression levels and continuing states are crucially important for the developmental maturation and function of natural regulatory T cells. Furthermore, FOXP3 binds to multiple transcription factors and histone acetyltransferase/deacetylase complexes with enzymatic activity. This is required for the inhibition of transcription activation of pro-inflammatory cytokines in T cells. And, FOXP3 protein post-translational modification, transcription complex assembly, and their modification enzyme activities are dynamically regulated by T cell receptors and inflammatory cytokine receptor signaling. Previous studies found that FOXP3 is a lysine acetylated protein, and the FOXP3 proline-rich N-terminus can directly recruit histidine acetyltransferase TIP60 (Tat interaction protein, 60 kDa), thereby mediating FOXP3 transcription inhibitory activity. (See Reference 6).

Proto-oncogene PIM1 is a constitutively active Ser/Thr kinase. It was first discovered as a collaborative oncogene with proto-oncogene MYC in lymphoma. Subsequently, this proto-oncogene expression was found in a variety of human cancer tissues, including pancreatic cancer, acute myeloid leukemia, and other malignant liver cancer. PIM1 is generally expressed in tissues or cells of spleen, thymus, bone marrow, pancreas, oral endothelial cells, etc., and is highly expressed in tumor cells. PIM1 has many important functions, including affecting programmed cell death, gene translation activation, cell signal transduction, etc.

IL-2 is a lymphocyte factor and is usually produced by T lymphocytes activated by lectin or antigens. It can allow proliferation of cytotoxic T cells, natural killer cells, and killer cells activated by lymphokine. It also enhances killing activity and promotes antibody and interferon secretions by lymphocytes, having the effects of anti-viral, anti-tumor, enhancing immune function, etc. Human recombinant IL-2 has been applied to the treatment of cancer, inflammation, acute infectious diseases, or chronic infectious diseases, respectively.

GITR is a member of tumor necrosis factor receptor protein family, also known as TNFRSF18. It's expression usually increases in the activated T cells. It is also an important cytokine in regulatory T cells and plays an important role in innate immunity. In vivo mouse gene knockout showed that GITR regulates CD3-driven T cell activation and programmed cell death. Other studies showed that use of activating GITR antibody can inhibit the function of regulatory T cells or stimulate the proliferation of regulatory T cells or effector T cells.

CTLA4 is a member of immunoglobulin family. It is usually expressed in helper T cell surface and transduces inhibitory signals to T cells. CTLA4 is an analogue of T cell co-stimulatory factor CD28 and can bind to CD80 and CD86 on antigen-presenting cell surface. CTLA4 is also an important cytokine in regulatory T cells, playing an important role in regulatory T cell function.

Therefore, in-depth studies of biochemical activity, physiological function, and molecular mechanism of the regulation of regulatory T cells and transcription factor FOXP3, IL-2, GITR, and CTLA4 are essential for therapeutic control of immune activity of human regulatory T cells and for immunotherapy based on regulatory T cells. This provides new research propositions and challenges in translating basic immunology research into clinical research, and in understanding of major human-specific infectious diseases.

### SUMMARY OF INVENTION

One of the main objects of the present invention is to provide use of phosphorylation pathway-related factors and agonists or antagonists thereof in the regulation of FOXP3 activity, IL-2 activity, GITR activity, and CTLA4 activity. Another object of the present invention is to provide use of these regulators through regulating the activities of FOXP3, IL-2, GITR, and CTLA4 to regulate regulatory T cell function. Other important objects of the present invention also include: providing use of proto-oncogene protein PIM1 and/or the coding sequence thereof, the agonists or the antagonists thereof in the preparation of compositions containing regulatory T cells or compositions for the regulation of regulatory T cell activity; methods of preparing compositions containing regulatory T cells; methods for the regulation of regulatory T cell activity; and reagents and kits used for the preparation of compositions containing regulatory T cells or compositions for the regulation of the regulatory T cell activity.

In a first aspect of the present invention, it provides use of phosphorylation pathway-related factors, agonists or antagonists thereof in the preparation of compositions for regulating regulatory T cell immune function, in which the phosphorylation pathway-related factors are selected from: proto-oncogene protein PIM1 and/or the coding sequence thereof. The regulation is achieved by regulating the activity of regulators of regulatory T cells. The regulators are selected from the group: FOXP3, IL-2, GITR, CTLA4, or a combination thereof.

In one embodiment of the present invention, the regulation of the activity of FOXP3, IL-2, GITR, and CTLA4 or a combination thereof is a positive regulation or a negative regulation, in which phosphorylation pathway-related factors or agonists thereof negatively regulate the activity of FOXP3, GITR, or CTLA4 and positively regulate IL-2 activity; and antagonists of phosphorylation pathway-related factors positively regulate the activity of FOXP3, GITR, or CTLA4 activity and negatively regulate IL-2 activity.

In another preferred embodiment, the proto-oncogene protein PIM1 or agonists thereof promote FOXP3 phosphorylation at S422 site.

In another embodiment of the present invention, the agonists are selected from: signals factors and/or the coding sequences thereof that induce PIM1 expression; the antagonists are selected from: anti-PIM1 antibody, shPIM1, antisense oligonucleotides targeting PIM1, chemical inhibitors of PIM1, such as see References 8-14, preferably 4-[3-(4-chlorophenyl)-2,1-benzisoxazol-5-yl]-2-pyrimidinamine, 3-cyano-4-phenyl-6-(3 bromo-6-hydroxy)phenyl-2(1H)-pyridinone, 2-hydroxy-3-cyano-4-phenyl-6-(3-bromo-6-hydroxyphenyl)pyridine, a racemic mixture of a pyridocarbazole-cyclopentadienyl Ruthenium complex, or other PIM1 inhibitors known in the art or those selected by methods known in the art, more preferably 4-[3-(4-chlorophenyl)-2,1-benzisoxazol-5-yl]-2-pyrimidinamine.

In another embodiment of the present invention, the agonists are selected from inflammatory cytokines, more preferably IL-4, IL-6, TCR, or the coding sequence thereof.

In another embodiment of the invention, the antagonist is kaempferol or a pharmaceutically acceptable salt thereof.

In a preferred embodiment, the inhibitors enhance GITR and CTLA4 mRNA expression, and inhibit IL-2 mRNA expression.

In another embodiment of the present invention, the use is using phosphorylation pathway-related factors or the agonists thereof in the preparation of compositions for the negative regulation of regulatory T cell immunomodulation.

In another embodiment of the invention, the compositions are used for the treatment and/or the prevention of diseases or symptoms related to dysfunction of regulatory T cell immunomodulation.

In another embodiment of the present invention, the diseases or the symptoms related to dysfunction of regulatory T cell immunomodulation are diseases or symptoms related to too high or too low regulatory T cell immunosuppressive function.

In one preferred embodiment, the diseases or the symptoms related to dysfunction of regulatory T cell immunomodulation are selected from: tumors or viral infections, preferably viral infections; inflammatory reaction, rheumatoid arthritis, organ transplantation, systemic lupus erythematosus, Crohn's disease or ulcerative colitis, infectious diseases, and preferably rheumatoid arthritis.

In one preferred embodiment, the diseases or the symptoms related to too high immunosuppressive function of regulatory T cells are selected from: tumors or viral infections, preferably viral infections.

In another preferred embodiment, the diseases or the symptoms related to too low immunosuppressive function of regulatory T cells are selected from: rheumatoid arthritis, organ transplantation, systemic lupus erythematosus, Crohn's disease, or ulcerative colitis, preferably rheumatoid arthritis.

In another preferred embodiment, the tumors are selected from: prostate cancer, breast cancer, liver cancer, gliomas, colorectal cancer, cervical cancer, non-small cell lung cancer, lung cancer, pancreatic cancer, stomach cancer, bladder cancer, skin cancer, striated muscle cancer, tongue squamous cell carcinoma, nasopharyngeal cancer, ovarian cancer, placenta cancer, neurogliomas, lymphoma, leukemia, colorectal cancer, or melanoma.

The inflammatory reaction is selected from: allergic inflammation, folliculitis, tonsillitis, pneumonia, hepatitis, nephritis, acne, asthma, autoimmune diseases, chronic inflammation, chronic prostatitis, glomerulonephritis, hypersensitivity, inflammatory intestinal diseases, pelvic inflammatory disease, reperfusion injury, rheumatoid arthritis, transplant rejection, vasculitis, or interstitial cystitis.

The infectious disease is selected from: plague, cholera, SARS, AIDS, viral hepatitis, polio, highly pathogenic avian influenza, measles, epidemic hemorrhagic fever, rabies, hepatitis B epidemic encephalitis, foot and mouth disease, dengue fever, anthrax, bacillary and amoebic dysentery, tuberculosis, typhoid and paratyphoid fever, epidemic cerebrospinal meningitis, whooping cough, diphtheria, neonatal tetanus, scarlet fever, brucellosis, gonorrhea, syphilis, leptospirosis, schistosomiasis, malaria, influenza, mumps, rubella, acute hemorrhagic conjunctivitis, leprosy, epidemic and endemic typhus, leishmaniasis, hydatid disease, filariasis, infectious diarrhea, or fungal infection, except cholera, bacillary and amoebic dysentery, typhoid, and paratyphoid.

In another embodiment of the invention, the compositions are pharmaceutical compositions, healthcare compositions, adjuvant compositions, or vaccine compositions.

In a preferred embodiment, the vaccines are selected from: vaccines for the treatment or the prevention of viral infections, bacterial infections, inflammation, parasitic infection, or tumors.

In a second aspect of the present invention, it provides compositions that regulate regulatory T cell immunomodulatory function, including:
(a) one or more phosphorylation pathway-related factors selected from the group: proto-oncogene protein PIM1 and/or the coding sequence thereof; and/or agonists or antagonists of the phosphorylation pathway-related factors;
(b) pharmaceutically, immunologically, or healthcare science acceptable carriers, in which the regulation is achieved by regulating the activity of regulatory factors of regulatory T cells, and the regulators are selected from the group: FOXP3, IL-2, GITR, CTLA4, or a combination thereof.

With regard to the phosphorylation pathway-related factors, the agonists or the antagonists thereof, use of the compositions, etc., they are described as that in the first aspect of the present invention, and will not be reiterated here.

In one embodiment of the present invention, the compositions are pharmaceutical compositions, healthcare compositions, vaccine compositions, or adjuvant compositions.

In a preferred embodiment, the compositions are used for the treatment or the prevention of diseases or symptoms related to the dysregulation of FOXP3, IL-2, GITR, and CTLA4 (i.e., activity too high or too low) or as vaccine adjuvant.

In another preferred embodiment, the compositions are used for the treatment or the prevention of diseases or symptoms related to dysfunction of regulatory T cell immunomodulation.

In another preferred embodiment, the compositions further include one or more other active substances capable of regulating FOXP3, IL-2, GITR, and CTLA4, preferably TCR or TGF-β.

In one preferred embodiment, the content of the regulators in the compositions are 0.05-99.5 wt %, preferably 0.1-95 wt %, more preferably 1-90 wt %, more preferably 5-80 wt %.

In another preferred embodiment, the compositions are injectable agents, tablet agents, granule agents, powder agents, or capsules.

In a third aspect of the present invention, it provides use of phosphorylation pathway-related factors, the agonists or antagonists thereof in the preparation of compositions that regulate the activities of FOXP3, IL-2, GITR, CTLA4, or a combination thereof, in which the phosphorylation pathway-related factors are selected from: proto-oncogene protein PIM1 and/or the coding sequence thereof.

In one embodiment of the present invention, the regulation of the activity of FOXP3, IL-2, GITR, and CTLA4, or the combination thereof is a positive regulation or a negative regulation, in which the phosphorylation pathway-related factors or the agonists thereof negatively regulate the activity of FOXP3, GITR or CTLA4 and positively regulate IL-2 activity. The antagonists of the phosphorylation pathway-related factors positively regulate the activity FOXP3, GITR, or CTLA4 activity and negatively regulate IL-2 activity.

With regard to the phosphorylation pathway-related factors, the agonists or the antagonists thereof, use of the compositions, etc., they are described as that in the first aspect of the present invention, and will not be reiterated here.

Accordingly, the present invention also provides compositions that regulate the activity of FOXP3, IL-2, GITR, CTLA4, or a combination thereof, including:
(a) one or more phosphorylation pathway-related factors selected from the group: proto-oncogene protein PIM1 and/or the coding sequence thereof; and/or the agonists or the antagonists of the phosphorylation pathway-related factors;
(b) pharmaceutically, immunologically, or healthcare science acceptable carriers.

With regard to the phosphorylation pathway-related factors, the agonists or the antagonists thereof, use of the compositions, etc., they are described as that in the first aspect and the second aspect of the present invention, and will not be reiterated here.

In other aspects of the present invention, they provide a method of regulating regulatory T cell immunomodulatory function through the regulation of FOXP3 activity, IL-2 activity, GITR activity, CTLA4 activity, or a combination thereof. The method includes administering to a subject in need thereof an effective amount of one or more substances selected from the group: (i) phosphorylation pathway-related factors selected from the group: PIM1 and/or the coding sequence thereof: (ii) agonists of the phosphorylation pathway-related factors, preferably: signal factors and/or the coding sequences thereof that induce PIM1 expression, more preferably inflammatory cytokines, more preferably IL-4, IL-6, TCR, or the coding sequence thereof; and/or (iii) antagonists of the phosphorylation pathway-related factors, preferably anti-PIM1 antibodies, shPIM1, antisense oligonucleotides targeting PIM1, chemical inhibitors of PIM1, or a combination thereof, for example, see References 8-14, more preferably 4-[3-(4-chlorophenyl)-2,1-benzisoxazol-5-yl]-2-pyrimidinamine, 3-cyano-4-phenyl-6-(3-bromo-6-hydroxy)phenyl-2(1H)-pyridinone, 2-hydroxy-3-cyano-4-phenyl-6-(3-bromo-6-hydroxyphenyl)pyridine, a racemic mixture of a pyridocarbazole-cyclopentadienyl Ruthenium complex, or other PIM1 inhibitors known in the art or can be selected using methods known in the art, most preferably 4-[3-(4-chlorophenyl)-2,1-benzisoxazoloxazol-5-yl]-2-pyrimidinamine.

In another embodiment of the present invention, the antagonist is kaempferol or a pharmaceutically acceptable salt thereof.

In another preferred embodiment, the present invention further provides methods for the treatment or the prevention of diseases or symptoms related to dysregulation of FOXP3 activity, IL-2 activity, GITR and CTLA4 activities. The methods include administering to a subject in need thereof an effective amount of one or more substances selected from the group: (i) phosphorylation pathway-related factors selected from the group: PIM1 and/or the coding sequence thereof: (ii) agonists of the phosphorylation pathway-related factors, preferably: signal factors and/or the coding sequences thereof that induce PIM1 expression, more preferably inflammatory cytokines, more preferably IL-4, IL-6, TCR, or the coding sequence thereof; and/or (iii) antagonists of the phosphorylation pathway-related factors, preferably anti-PIM1 antibodies, shPIM1, antisense oligonucleotides targeting PIM1, chemical inhibitors of PIM1, or a combination thereof, for example, see References 8-14, more preferably 4-[3-(4-chlorophenyl)-2,1-benzisoxazol-5-yl]-2-pyrimidinamine, 3-cyano-4-phenyl-6-(3-bromo-6-hydroxy)phenyl 2(1H)-pyridinone, 2-hydroxy-3-cyano-4-phenyl-6-(3-bromo-6-hydroxyphenyl)pyridine, a racemic mixture of a pyridocarbazole-cyclopentadienyl Ruthenium complex, or other PIM1 inhibitors known in the art or can be selected using methods known in the art, most preferably 4-[3-(4-chlorophenyl)-2,1-benzisoxazoloxazol-5-yl]-2-pyrimidinamine.

In another preferred embodiment, the method is further used for enhancing vaccine immunogenicity.

In another aspect the present invention, it provides use of phosphorylation pathway-related factors, the agonists or the antagonists thereof in the preparation of compositions containing regulatory T cells or compositions that regulate the regulatory T cell activity, in which the phosphate pathway-related factors are selected from: proto-oncogene protein PIM1 and/or the coding sequence thereof.

In some embodiments, the preparations use the phosphorylation pathway-related factors, the agonists or the antagonists thereof to regulate the activity of regulators of regulatory T cells. The regulators are selected from the group: FOXP3, IL-2, GITR, CTLA4, or a combination thereof.

In other embodiments, the regulation of the activity of FOXP3, IL-2, GITR, CTLA4, or a combination thereof is a positive regulation or a negative regulation, in which the phosphorylation pathway-related factors or the agonists thereof negatively regulate the activity of FOXP3, GITR or CTLA4 and positively regulate IL-2 activity. The antagonists of the phosphorylation pathway-related factors positively regulate FOXP3, GITR, or CTLA4 activity and negatively regulate IL-2 activity.

In other embodiments, the agonists are selected from: signal factors and/or the coding sequences thereof that induce PIM1 expression; the antagonists are selected from: anti-PIM1 antibodies, shPIM1, antisense oligonucleotides targeting PIM1, chemical inhibitors of PIM1, for example, see References 8-14, preferably 4-[3-(4-chlorophenyl)-2,1-benzisoxazol-5-yl]-2-pyrimidinamine, 3-cyano-4-phenyl-6-(3-bromo-6-hydroxy)phenyl-2(1H)-pyridinone, 2-hydroxy-3-cyano-4-phenyl-6-(3-bromo-6-hydroxyphenyl)pyridine, a racemic mixture of a pyridocarbazole-cyclopentadienyl Ruthenium complex.

In other embodiments, the agonists are selected from inflammatory cytokines, more preferably IL-4, IL-6, TCR, or the coding sequence thereof.

In other embodiments, the antagonist is kaempferol or a pharmaceutically acceptable salt thereof.

In other embodiments, starting materials for the preparation of the regulatory T cells include CD4+ T cells, preferably CD4+ CD45RA+ natural T cells.

In a preferred embodiment, the regulatory T cells are prepared from CD4+ T cells.

In another preferred embodiment, when phosphorylation pathway-related factors or the agonists thereof are used, FOXP3 expression decreases in the compositions, immunosuppressive activity of regulatory T cells decreases, or FOXP3 expression or immunosuppressive activity of regulatory T cells in subjects that received the compositions is lower than that prior to receiving the compositions.

In another preferred embodiment, when antagonists of phosphorylation pathway-related factors are used, FOXP3 expression increases in the compositions, i.e., immunosuppressive activity of regulatory T cells increases, or FOXP3 expression or immunosuppressive activity of regulatory T cells in subjects that received the compositions is higher than that prior to receiving the compositions.

In another preferred embodiment, the regulatory T cells are CD4+ CD25+CD 127-low expressing T cells.

In other embodiments, the compositions are used for the treatment and/or the prevention of diseases or symptoms related to dysfunction of regulatory T cell immunomodulation.

In other embodiments, the diseases or the symptoms related to dysfunction of regulatory T cell immunomodulation are diseases or symptoms related to regulatory T cells having too high or too low immunosuppressive function.

In other embodiments, the compositions are pharmaceutical compositions, healthcare compositions, vaccine compositions, or adjuvant compositions.

In another aspect of the present invention, it provides a method for the preparation of compositions containing regulatory T cells, the method include:
(a) providing starting materials containing CD4+ T cells;
(b) contacting the starting materials with phosphorylation pathway-related factors, the agonists or the antagonists thereof to obtain regulatory T cells, or compositions containing regulatory T cells, in which the phosphorylation pathway-related factors are selected from: proto-oncogene protein PIM1 and/or the coding sequence thereof.

In a preferred embodiment, the starting materials are obtained from: WBC isolates, blood (e.g., peripheral blood, umbilical cord blood), lymphatic system, bone marrow, spleen.

In another preferred embodiment, the contacting results in changes in the activity of FOXP3, IL-2, GITR, CTLA4 or a combination thereof selected.

In another preferred embodiment, the regulation of the activity of FOXP3, IL-2, GITR, CTLA4, or a combination thereof is a positive regulation or a negative regulation, in which the phosphorylation pathway-related factors or the agonists thereof negatively regulate FOXP3, GITR or CTLA4 activity and positively regulate IL-2 activity. The antagonists of the phosphorylation pathway-related factors positively regulate FOXP3, GITR, or CTLA4 activity and negatively regulate IL-2 activity.

In another preferred embodiment, the agonists are selected from: signal factors and/or the coding sequences thereof that induce PIM1 expression; the antagonists are selected from: anti-PIM1 antibodies, shPIM1, antisense oligonucleotides targeting PIM1, chemical inhibitors of PIM1, for example, see References 8-14, preferably 4-[3-(4-chlorophenyl)-2,1-benzisoxazol-5-yl]-2-pyrimidinamine, 3-cyano-4-phenyl-6-(3-bromo-6-hydroxy)phenyl-2(1H)-pyridinone, 2-hydroxy-3-cyano-4-phenyl-6-(3-bromo-6-hydroxyphenyl)pyridine, a racemic mixture of a pyridocarbazole-cyclopentadienyl Ruthenium complex.

In another preferred embodiment, the agonists are selected from inflammatory cytokines, more preferably IL-4, IL-6, TCR, or the coding sequence thereof.

In another preferred embodiment, the antagonist is kaempferol or a pharmaceutically acceptable salt thereof.

In other embodiments, the methods further optionally include one or more steps selected from the group:

step (a′) before step (b), purifying or isolating the starting materials, preferably purifying or isolating CD4+ T cells;
step (c) after step (b), purifying or isolating the regulatory T cells or cells in the compositions containing the regulatory T cells.

In another aspect of the present invention, it provides methods of regulating regulatory T cell activity, the methods include: contacting samples or objects containing CD4+ T cells with phosphorylation pathway-related factors, the agonists or the antagonists thereof, or the compositions prepared by the methods of the present invention.

In a preferred embodiment, the samples or the objects are selected or obtained from: WBC isolates, blood (e.g., peripheral blood, umbilical cord blood), lymphatic system, bone marrow, spleen; mammals, preferably human.

In another aspect of the present invention, it provides reagents or kits used for the preparation of compositions containing regulatory T cells or compositions for the regulation of regulatory T cell activity, including: (a) one or more phosphorylation pathway-related factors selected from the group: proto-oncogene protein PIM1 and/or the coding sequence thereof; and/or agonists or antagonists of the phosphorylation pathway-related factors; (b) pharmaceutically, immunologically, or healthcare science acceptable carriers.

In other embodiments, the regulation is achieved by regulating the activity of regulatory factors of regulatory T cells, in which the regulators are selected from the group: FOXP3, IL-2, GITR, CTLA4, or a combination thereof.

In other embodiments, the antagonist is kaempferol or a pharmaceutically acceptable salt thereof.

In a further aspect of the present invention, it provides methods of selecting substances that regulate regulatory T cell activity, the methods include detecting FOXP3 protein stability or post-translational modification enzyme (such as PIM1) activity, that is, the substances that regulate the regulatory T cell activity are selected by detecting FOXP3 phosphorylation induced by PIM1 or FOXP3 protein stability.

In one preferred embodiment, the phosphorylation site is serine 422 of FOXP3.

In another preferred embodiment, the methods include selecting PIM1 inhibitors used for the preparation of compounds of regulatory T cells, from which to obtain immunomodulating drugs.

In another preferred embodiment, a decrease of PIM1-induced FOXP3 phosphorylation or an increase of FOXP3 protein stability indicates the test substances can be used to positively regulate the immunosuppressive activity of regulatory T cells. In contrast, an increase of PIM1-induced FOXP3 phosphorylation or a decrease of FOXP3 protein stability indicates the test substances can be used to negatively regulate the immunosuppressive activity of regulatory T cells.

Other aspects of the present invention, due to the disclosure herein, would be obvious to a person skilled in the art.

### BRIEF DESCRIPTION OF DRAWINGS

FIGS. 1A-1C show that FOXP3 binds to PIM1 and is phosphorylated by PIM1.

FIG. 1A shows immunoblot detection using corresponding antibodies following co-immunoprecipitation by FLAG antibody in HEK 293T cells co-transfected with HA-FOXP3 and FLAG-PIM1, collected after 48 h, and lysed in RIPA lysis buffer.

FIG. 1B shows immunoblot analysis using Ser422 phosphorylation-specific antibodies and other corresponding antibodies following immunoprecipitation by HA antibody in HEK 293T cells co-transfected with HA-FOXP3 or its Ser422A mutant and FLAG-PIM1 or its enzymatic activity-deficient mutant K67M (respectively: HA-S422A FOXP3; FLAG-K67M PIM1+HA-S422A FOXP3; HA-S422A FOXP3+FLAG-PIM1; HA-FOXP3; HA-FOXP3+FLAG-K67M PIM1; HA-FOXP3+FLAG-PIM1), collected and lysed after 48 h.

FIG. 1C shows FPLC-purified MBP-FOXP3 and His-PIM1 and its K67M mutant, which were expressed by prokaryotic cells, after in vitro reaction in the presence of ATP, were analyzed by immunoblot. Total proteins in the reaction system were subjected to SDS-PAGE, and then shown under ultraviolet light after Lumitein™ protein gel staining.

FIG. 2 shows inflammatory cytokines IL-6 and IL-4 can induce PIM1 expression in SZ4.03 lymphocyte line that highly expresses FOXP3.

SZ4.03 cells were treated with IL-6 (20 ng/ml) or IL-4 (30 ng/ml), respectively. After 0 h, 1 h, 2 h, and 4 h, cells were collected and lysed by boiling directly in SDS-Loading Buffer. Samples were then analyzed by immunoblot.

FIG. 3 shows PIM1 inhibits FOXP3 binding to chromatin.

HA-FOXP3 and FLAG-PIM1 were co-transfected into HEK 293T cells. After 48 h, cells were collected and cytoplasm (Cy), nucleus (Nu), and chromatin (Chr) were isolated. The isolated samples were analyzed by immunoblot. The data represent two independent test results.

FIGS. 4A-4E show PIM1 negatively regulates the transcription regulatory activity of FOXP3 transcription factor.

FIG. 4A shows SZ4.03 cell line that highly expresses FOXP3 was infected with lentivirus packaged with a negative control shRNA (shCK) and PIM1 shRNA (shPIM1), selected by puromycin (50 ng/ml), and treated by DMSO or PMA/Inomycin to activate T cell activity. After 4 h, total RNA was extracted and converted to cDNA. PIM1 mRNA expression levels were then analyzed by quantitative PCR.

FIGS. 4B-C show FOXP3 positively regulates the mRNA expression levels of target genes (GITR and CTLA4).

FIG. 4D shows FOXP3 negatively regulates the mRNA expression levels of target gene (IL-2).

FIG. 4E shows the mRNA expression levels of non-FOXP3 target gene (STUB1).

FIGS. 5A-5B show PIM1 inhibitors significantly increase cell levels of CLTA4+ FOXP3+ Treg and CD25+ FOXP3+ Treg.

FIG. 5A shows human CD4+ CD25+ CD127¹° ″ natural Treg (nTreg) after sorted by BD FACS Aria II flow sorter and expanded through Bead-CD3/CD28 and IL-2 (500 U/ml). After removal of Beads, DMSO or PIM1 inhibitor (100 nM) was added and treated for 24 h, followed by APC-FOXP3 and PE-CD25 antibody staining for flow cytometry analysis.

FIG. 5B shows the expanded nTreg after Beads removal and rested for 24 h, DMSO or PIM1 inhibitor (100 nM) was then added and treated for 48 h, followed by APC-FOXP3 and PE-CD25 antibody staining for flow cytometry analysis.

FIG. 6 shows PIM1 inhibitors can enhance immunosuppressive activity of nTreg.

The expanded nTreg were mixed with human CFSE-labeled PBMC (2×10⁵ cells/well) in U-shaped wells of 96-well plate at corresponding proportion. 5000 T cell dynabeads conjugated with CD3/CD28 antibody were added. Cell proliferation levels were detected after two groups of cells treated with DMSO and PIM1 inhibitors, respectively, were incubated for five days.

FIG. 7 shows PIM1 inhibitors promote FOXP3+ and CTLA4+ nTreg cell expansion.

500 U/ml of IL-2 was added to 2×10′ nTreg cells. nTreg cells were expanded using CD3/CD28 T cell dynabeads and cells at 3:1 ratio. After two weeks, part of expanded nTreg cells were collected for FACS detection. Dynabeads were removed from the remaining cells. After 48 h of rest, FOXP3 and CTLA4 cells were collected again by flow cytometry. The working concentrations of Rapamycin and PIM1 inhibitors are both 100 nM.

FIG. 8 shows PIM1 protein expression knockout can enhance FOXP3 transcription regulatory activity.

Primary nTreg cells were activated with 500 U/ml of IL-2 and CD3/CD28 T cell dynabeads and cells at 3:1 ratio for 4 h, and, respectively, infected with lentivirus encoding PIM1, PIM2, and PIM3 shRNA overnight. Continued incubation for one week, the mRNA levels of FOXP3 and its target gene CTLA4 were detected by quantitative PCR.

FIGS. 9A-9B show kaempferol inhibits FOXP3 S422 phosphorylation in a dose-dependent manner.

FIG. 9A shows the protein levels of pS422-FOXP3, FOXP3, and α-tubulin in cell lysates detected directly by immunoblot. Myc-FOXP3 and FLAG-PIM1 were co-transfected into HEK293T cells. After 36 h, cells were treated, respectively, with 50 nM, 10 nM, 2 nM, 0.4 nM of PIM1 inhibitor and 50 μM, 10 μM, 2 μM, 0.4 μM, 0.08 μM of kaempferol for 12 h. Cells were collected at 48 h after cell transfection.

FIG. 9B shows the effect of small molecule inhibitor on FOXP3 S422 phosphorylation using ImageJ software analyzing FIG. A.

FIGS. 10A-10B show kaempferol can specifically inhibit FOXP3 S422 phosphorylation induced by PIM1.

FIG. 10A shows the protein levels of pS422-FOXP3, FOXP3, and β-tubulin in cell lysates detected directly by immunoblot. Myc-FOXP3, FLAG-PIM1, FLAG-PIM2, and FLAG-PIM3 were co-transfected into HEK293T cells. 36 h later, cells were treated with 50 μM of kaempferol for 12 h. Cells were collected 48 h after cell transfection.

FIG. 10B shows small molecule inhibitor can specifically inhibit FOXP3 S422 phosphorylation induced by PIM1 using ImageJ software analyzing FIG. 10A.

FIG. 11 shows kaempferol can inhibit histone H3 S10 phosphorylation induced by PIM1.

FLAG-PIM1 was transfected into HEK293T cells. 36 h later, cells were treated with 50 μM of kaempferol for 12 h. 48 h after cell transfection, cells were treated in whole cell lysis solution. The levels of phosphorylated (Phospho)-histone H3S10, histone H3, and PIM1 were detected directly by immunoblot.

FIG. 12 shows kaempferol only inhibits FOXP3 S422 phosphorylation and histone H3 S10 phosphorylation induced by PIM1.

FLAG-PIM1 was transfected and MYC-FOXP3 and FLAG-PIM1 co-transfected into HEK293T cells. After 36 h, HEK293 was used as blank control group and each group was treated with 50 μM of kaempferol for 12 h. After 48 h, cells collected and lysed by cell lysis buffer. The levels of pS422-FOXP3, FOXP3, phosphorylated-histone H3 (Ser10), histone H3, and GAPDH were detected directly by immunoblot.

FIG. 13 shows kaempferol can inhibit FOXP3 protein degradation induced by PMA/LPS.

HA-FOXP3-Jurkat stable expression cells were treated with PMA (25 ng/ml) and LPS (1 ng/ml). 50 μM kaempferol, 100 nM PIM1 inhibitors (specifically of 4-[3-(4-chlorophenyl)-2,1-benzisoxazol-5-yl]-2-pyrimidinamine), and 10 μg/ml PI3K inhibitor (LY294002) were added, and DMSO as a control. Cells were collected 24 h later. The levels of FOXP3 and GAPDH were detected directly by immunoblot.

FIG. 14 shows kaempferol enhances the expression of FOXP3 and CTLA4 in the induced Treg.

Human CD4+ CD45RA+ natural T cells (i.e., natural T cells rather than Treg cells) were sorted by BD FACS Aria II flow sorter and expanded by Bead-CD3/CD28 (2:1), IL-2 (100 U/ml), and TGF-β (1 ng/ml, 3 ng/ml, and 5 ng/ml) for 5 days. DMSO or kaempferol (50 uM) was added and treated for 48 h. Flow cytometry analysis was performed after PE-FOXP3 and APC-CTLA4 antibody staining.

### DETAILED DESCRIPTION

The inventors believe that FOXP3 can be regulated in a series of time and space, including post-transcriptional modification, translation or modification, and protein-protein interactions, etc. Among them, a class of factors (including enzymes, small molecule compounds, etc.) serve as FOXP3 negative regulators capable of down-regulating FOXP3 activity under different signaling stimuli, thereby regulating the entire immune system. Through long-term and in-depth studies, the inventors found that phosphorylation pathway-related factors can affect FOXP3 binding to chromatin, thereby regulating FOXP3 transcription regulatory activity on its target genes. These phosphorylation pathway-related factors are FOXP3 negative regulators—including PIM1 protein and its inflammation inducing signals. Thus, also in the present application, the inventors further provide use of proto-oncogene protein PIM1 and/or the coding sequence thereof, the agonists or the antagonists thereof in the preparation of compositions containing regulatory T cells or compositions that regulate regulatory T cell activity; methods of preparing compositions containing regulatory T cells; methods of regulating regulatory T cell activity; and a reagent or a kit used in the preparation of compositions containing regulatory T cells or compositions that regulate regulatory T cell activity.

Specifically, the inventors, in study prior to the present invention, have constructed cell lines stably expressing FOXP3 and have purified FOXP3 protein under different signaling stimuli. The purified proteins were analyzed by mass spectrometry, thereby identifying multiple Ser/Thr/Tyr phosphorylation sites in FOXP3 protein sequences. In the present study, the inventors performed a large number of screening tests and found FOXP3-S422 is a potential phosphorylation site for PIM1. Further experiments proved that PIM1 protein can interact with FOXP3 and specifically phosphorylates FOXP3 Ser422 in vivo and in vitro. Moreover, further studies found, under inflammatory cytokine stimulation, such as the stimulation of IL-6, IL-4, etc., the in vitro expanded natural regulatory T cells (nTreg) can rapidly up-regulate PIM1 protein expression levels. Thus, it is possible that PIM1 plays an important regulatory role in nTreg activity.

Recently, studies found that PIM1 plays an important role in the transformation of normal cells to become cancerous cells promoted by collaborative proto-oncogene encoded MYC protein. PIM1 interacts with MYC protein and is recruited to the target genes of MYC transcription factor and phosphorylates Ser10 of histone H3, thereby activating the transcription of target genes. We found the phosphorylation site in FOXP3 sequence by PIM1 is located within Forkhead domain, which is an important domain that determines its interaction with DNA or chromatin. Thus, we believe it is possible that PIM1 affects FOXP3 binding to genomic DNA, thereby regulating nTreg physiological activity. Experimental results are consistent with such predications. PIM1 overexpression significantly inhibits FOXP3 binding to chromatin. PIM1 knockout in FOXP3+ Treg significantly enhances transcriptional regulatory activity of FOXP3 on its target genes: including promoting the transcription of FOXP3-positively regulated genes (CTLA4, GITR, CD25, etc.) and inhibiting the transcription of FOXP3-negatively regulated gene (IL-2). Using PIM1 kinase-specific inhibitors to treat the expanded nTreg cells in vitro can significantly increase the levels of CD25+ FOXP3+ Treg and CTLA4+FOXP3+ Treg. Also, in vitro inhibition tests showed PIM1 inhibitors can significantly enhance the immunosuppressive activity of nTreg. Thus, we are confident that PIM1 regulates FOXP3 transcription regulatory activity and inhibits immunosuppressive activity of nTreg by modifying FOXP3 phosphorylation.

Previous studies found that increasing FOXP3 expression levels alone cannot enhance immunosuppressive activity of nTreg, suggesting that the exertion of FOXP3 transcription regulatory activity requires the regulation of other signals. Our research findings provide a possible pathway for this. It is possible to significantly increase immunosuppressive activity of FOXP3+ Treg by up-regulating FOXP3 expression levels and, simultaneously, inhibiting PIM1 expression levels or its activity.

Based on the above, the inventors in the present invention, revealed, for the first time, the effects of FOXP3 phosphorylation pathway-related factor—PIM1 kinase and inflammatory cytokines, its expression inducers, on the regulatory mechanisms of FOXP3+ Treg immune activity and the regulation of regulatory T cell activity. Identification and application of these regulators provide new ways for the treatment and/or the prevention of diseases or symptoms (e.g., cancer, autoimmune diseases, etc.) related to dysregulation of FOXP3+ Treg activity by regulating immunosuppressive activity of FOXP3+ Treg cells; and for the preparation of regulatory T cells or regulating its activity, and drug selection. Meanwhile, it also provides use of these regulators as vaccine adjuvant and new methods for improving immunogenicity of viral infection.

On this basis, the inventors have completed the present invention.

Phosphorylation Pathway-Related Factors, the Agonists or the Antagonists Thereof

As used herein, the term “regulators” or “activity regulators” refers to phosphorylation pathway-related factors or the agonists or the antagonists thereof. They can affect FOXP3 binding to chromatin through FOXP3 phosphorylation, thereby regulating FOXP3 transcription regulatory activity on its target genes, regulating immunosuppressive activity of Treg. Thus, they can be used for the treatment and/or the prevention of diseases or symptoms related to dysregulation of FOXP3+ Treg cell activity (e.g., cancer, autoimmune disease), or used as vaccine adjuvant to increase immunogenicity of viral infection.

As used herein, the term “phosphorylation pathway-related factors” refers to activity factors that affect the activity of its downstream factors or processes (such as FOXP3 transcription regulatory activity on its target genes, regulatory activity of regulatory T cells on immune function) through affecting phosphorylation pathways. In the present invention, phosphorylation pathway-related factors include: PIM1 protein and/or coding sequence thereof.

As used herein, PIM1 protein refers to PIM1 proto-oncogene protein, which is a constitutively active Ser/Thr kinase. It was first discovered in lymphoma as a cooperative oncogene with MYC proto-oncogene. PIM1 sequences known in the art include, but not limited to: Entrez Gene: 5292.

As used herein, “agonists” of phosphorylation pathway-related factors refer to substances that can promote the expression of phosphorylation pathway-related factors or enhance the activity of phosphorylation pathway-related factors. In the present invention, they may also refer to as “agonists,” e.g. PIM1 agonists. PIM1 agonists used in the present invention include, but are not limited to: inflammatory cytokines IL-6, IL-4; TCR and/or the coding sequences thereof. Broadly speaking, PIM1 agonists also include substances transferred to body, tissues or cells to enhance PIM1 levels or activity, such as PIM1 transgene and/or its carriers.

As used herein, “antagonists” of phosphorylation pathway-related factors refer to substances that can inhibit the expression of phosphorylation pathway-related factors or reduce the activity of phosphorylation pathway-related factors. In the present invention, they may also refer to as “inhibitors.” Antagonists useful in the present invention include, but are not limited to, the following PIM1 antagonists: anti-PIM1 antibodies, shPIM1, antisense oligonucleotides targeting PIM1, chemical inhibitors of PIM1, for example, see References 8-14, such as: PIM1 inhibitors: 4-[3-(4-Chlorophenyl)-2,1-benzisoxazol-5-yl]-2-pyrimidinamine; 3-Cyano-4-phenyl-6-(3-bromo-6-hydroxy)phenyl-2(1H)-pyridinone, 2-Hydroxy-3-cyano-4-phenyl-6-(3-bromo-6-hydroxyphenyl)pyridine; a racemic mixture of a pyridocarbazolo-cyclopentadienyl Ruthenium complex; or other PIM1 inhibitors, e.g., kaempferol, selected according to the methods of the present invention or selected by using methods known in the art. One skilled in the art can employ methods known in the art to prepare anti-PIM1 antibodies, shPIM1, antisense oligonucleotides targeting PIM1, or to identify chemical inhibitors of PIM1.

The proteins or polypeptides of the present invention can be purified natural products, or chemically synthesized products, or produced from prokaryotic or eukaryotic hosts (e.g., bacterial, yeast, higher plant, insect, and mammalian cells) using recombination techniques.

It should be understood that the above definitions of the terms in the present invention also include conservative polypeptide variants or their homologous polypeptides of the proteins and polypeptides. In one embodiment of the present invention, the proteins or polypeptides are derived from human or other eukaryotes, such as mouse, rat, cow, or monkey, and they are highly conserved among them.

Protein or polypeptide variants of the present invention include (but are not limited to): one or more (usually 1-50, preferably 1-30, more preferably 1-20, most preferably 1-10, such as 1, 2, 3, 4, 5, 6, 7, 8, 9, or 10) amino acid deletions, insertions, and/or substitutions, and one or several (usually 20 or less, preferably 10 or less, more preferably 5 or less) amino acid additions at the C-terminus and/or the N-terminus. For example, in the art, when substituting with functionally close or similar amino acids, the functions of proteins or polypeptides usually do not change. As another example, one or several amino acid additions at the C-terminus and/or the N-terminus usually also do not change the functions of proteins or polypeptides. One skilled in the art can easily determine these variations, which do not affect the activity of proteins or polypeptides, based on common knowledge in the art and/or routine experimentation.

As used herein, the term “coding sequences” refers to sequences encoded by PIM1 proto-oncogene of the present invention, or highly homologous sequences thereof, or molecules that hybridize with the sequences under stringent conditions, or gene family molecules having high homology.

As used herein, the term “stringent conditions” refers to: (1) hybridization and washing at relatively low ionic strength and relatively high temperatures, such as 0.2×SSC, 0.1% SDS, 60° C.; or (2) hybridization with denaturants, such as 50% (v/v) formamide, 0.1% calf serum/0.1% Ficoll, 42° C., etc.; or (3) hybridization occurs only when the identity between two sequences is at least 50%, preferably 55% or more, 60% or more, 65% or more, 70% or more, 75% or more, 80% or more, 85% or more, or 90% or more, more preferably 95% or more.

Full-length coding sequences or fragments thereof of the present invention can be obtained generally by PCR amplification, recombination, or synthesis methods. For PCR amplification, primers can be designed based on the related nucleotide sequences disclosed in the present invention, especially open reading frame sequences. Commercially available cDNA libraries or cDNA libraries prepared according to conventional methods known by one skilled in the art can be used as template to amplify and obtain relevant sequences. If sequences are relatively long, two or more PCR amplifications are often necessary, and then splice together the amplified fragments in correct order.

Compositions and Treatment or Prevention of Diseases

Regulators of the present invention can be used in the preparation of therapeutic or prophylactic pharmaceutical compositions, healthcare compositions or vaccine compositions, or adjuvant compositions.

Accordingly, in another aspect, the present invention also provides a composition, which includes (a) safe and effective amount of regulators of the present invention; and (b) pharmaceutically acceptable carriers or excipients. Amount of regulators of the present invention is usually 10 μg-100 mg/dose, preferably 100-1000 μg/dose.

As used herein, the term “effective amount” refers to amount of therapeutic agent for treating, ameliorating, or preventing target diseases or conditions, or amount showing detectable therapeutic or prophylactic effect. Precise effective amount for a subject depends on the object's size and health, nature and extent of disease, and therapeutic agents and/or therapeutic agent combination selected for administering. Therefore, it is not useful to pre-specify an accurate effective amount. However, for a given condition, routine experimentation can be used to determine an effective amount. Clinicians can determine that.

Pharmaceutical compositions may also contain pharmaceutically acceptable carriers. The term “pharmaceutically acceptable carriers” refers to carriers used in the administration of therapeutic agent. This term refers to some pharmaceutical carriers: they themselves do not induce antibody production harmful to individuals, who received the compositions, and do not have undue toxicity after the administration of compositions. These carriers are known to one skilled in the art. Full discussion of pharmaceutically acceptable excipients can be found in Remington's Pharmaceutical Sciences (Mack Pub. Co., NJ 1991). This type of carriers include (but are not limited to): saline, buffer, dextrose, water, glycerol, ethanol, adjuvant, and a combination thereof.

Pharmaceutically acceptable carriers in therapeutic compositions may contain liquids, such as water, saline, glycerol, and ethanol. In addition, auxiliary substances may also be present in these carriers, such as wetting agents or emulsifying agents, pH buffering substances, etc. Moreover, immunological compositions may further contain immune adjuvant.

Typically, therapeutic compositions may be prepared as injectable agents, e.g., liquid solutions or suspensions; and may also be prepared as solid form suitable for dissolving into solutions or suspensions prior to injection.

Once compositions of the present invention are prepared, they can be directly administered to subjects. Subjects for prevention or treatment can be animals; especially human.

Therapeutic or prophylactic pharmaceutical compositions (including vaccines) of the present invention containing regulators of the present invention may be administered orally, subcutaneously, intradermally, intravenously, etc. applications. Therapeutic agent dosage regimen may be single dose regimen or multiple dose regimens.

Compositions of the present invention may be used for regulating regulatory T cells, thereby treating or preventing diseases caused by immune dysregulation. When down-regulation of patient's immune response is required, compositions containing phosphorylation pathway-related factors of the present invention or agonists thereof may be used. When up-regulation of patient's immune response is required, compositions containing inhibitors of phosphorylation pathway-related factors may be used. Diseases requiring down-regulation of patent's immune response in treatment include, but are not limited to: autoimmune diseases, organ transplantation. Diseases requiring up-regulation of patent's immune response in treatment include but are not limited to: cancer, viral infection. The up-regulation may be achieved by inhibiting regulatory Treg cell function. The down-regulation may be achieved by enhancing regulatory Treg cell function.

### Main Advantages of the Present Invention

(1) The present invention reveals, for the first time, phosphorylation pathway-related factor—PIM1 affects FOXP3 binding to chromatin by phosphorylating FOXP3, and, in turn, regulates FOXP3 transcriptional regulatory activity on its target genes.

(2) Determination and application of regulators of the present invention provide a new way to regulate FOXP3 transcriptional regulatory activity on its target genes by phosphorylating FOXP3, thereby treating and/or preventing diseases or conditions (e.g., cancer, autoimmune diseases, etc.) related to dysregulation of FOXP3, IL-2, and/or IFN-γ activity.

(3) The present invention further provides use of PIM1 and/or its coding sequences, the agonists or the antagonists thereof in the preparation of compositions containing regulatory T cells or compositions regulating regulatory T cell activity; methods of preparing compositions containing regulatory T cells; methods of regulating regulatory T cell activity; and reagents or kits used in the preparation of compositions containing regulatory T cells or compositions regulating regulatory T cell activity.

### EXAMPLES

The following specific embodiments further illustrate the present invention. It should be understood that these embodiments are merely used to illustrate the present invention and are not intended to limit the scope of the present invention. One skilled in the art may make suitable modifications and changes to the present invention. These modifications and changes, however, are within the scope of the present invention.

Conventional methods in the art may be used for experimental methods not specified in the following examples, such as those referenced in “Molecular Cloning, A Laboratory Manual” (third edition, New York, Cold Spring Harbor Laboratory Press, 1989), or in accordance with conditions recommended by the suppliers. DNA sequencing methods are conventional methods in the art, and tests can also be provided by commercial companies.

Unless otherwise indicated, percentages and parts are calculated by weight. Unless otherwise defined, all professional and scientific terms have identical meanings well known to one skilled in the art. In addition, any methods and materials similar or equivalent to the disclosures can be applied to the methods of the present invention. Preferred embodiments of methods and materials in the disclosure are used for demonstration purposes only.

### Example I

### Materials and Methods

1. Plasmids and Vectors:

Referring to Reference 4, cloning and constructing N-terminal FLAG-tagged FOXP3, PIM1, and other plasmids (pIPFLAG2 and pIPFHA2 are eukaryotic expression vectors; pET28a and pET21-MBP prokaryotic expression vectors) and lentiviral packaging vector plasmids (FUGW, dR8.9, and VSV-G).

PIM1 gene was obtained from amplifying human peripheral blood mononuclear cell cDNA library. Primers were designed according to the sequences downloaded from Genbank as follows:

| | SEQ ID | |
|---|---|---|
| Primer | NO: | Sequence |
| PIM1-upstream | 1 | 5′-GAGGAATTCGATGCTCTTGTCCAAA |
| primer | | ATCAACTC-3′ |
| PIM1-downstream | 2 | 5′-GAGGGGAGGACCTGCCAGAAAG-3′ |
| primer | | |

2. Antibodies:

Flag antibodies (M2) were purchased from Sigma. FOXP3 antibodies (hFOXY) were purchased from eBioscience. PIM1 antibodies were purchased from Santa Cruz. pS422-FOXP3 antibodies were custom made by Abmart Inc. HRP-conjugated anti-mouse secondary antibodies were purchased from Promega.

3. Cells and Treatments:

Human HEK293T cells (purchased from Chinese Academy of Sciences Cell Bank (Catalog Number: GNHu17)) were cultured in DMEM (Dulbecco's modified Eagle's medium) containing 10% FBS, 100 units/ml penicillin-streptomycin, at 37° C. under 5% CO₂.

SZ4.03 cells (lymphocyte cell line that highly expresses FOXP3, kept in the present laboratory; also can be routinely obtained by one skilled in the art according to methods described in Reference 7) were cultured in 1640 culture medium containing 10% FBS, 100 units/ml penicillin-streptomycin, 100 units/ml non-essential amino acids, and 100 units/ml sodium pyruvate, at 37° C. under 5% CO₂.

Cell transfection: Lipofection was performed using Lipofectamin 2000 (Invitrogen) (performed according to operating instructions). Samples were collected 48 h after transfection for analysis.

Human embryonic kidney cell line HEK293 were co-transfected with FUGW-TAP-FOXP3, dR8.9, and VSV-G. Culture supernatants were collected 48 h and 72 h after transfection.

shPIM1 primer sequences were designed as follows:

| | SEQ ID | |
|---|---|---|
| Primer | NO: | Sequence |
| ShPIM1 primer 1 | 3 | CCGGGATACTCTCTTCTTCTCATAG |
| | | CTCGAGCTATGAGAAGAAGAGAGTA |
| | | TCTTTTT |
| ShPIM1 primer 2 | 4 | AATTAAAAAGATACTCTCTTCTTCT |
| | | CATAGCTCGAGCTATGAGAAGAAGA |
| | | GAGTATC |

shCK primer sequences were designed as follows:

| | SEQ ID | |
|---|---|---|
| Primer | NO: | Sequence |
| ShCK primer 1 | 5 | CCGGTCAACAAGATGAAGAGCACCAA |
| | | CTCGAGTTGGTGCTCTTCATCTTGTT |
| | | GTTTTTG |
| ShCK primer 2 | 6 | AATTCAAAAACAACAAGATGAAGAGC |
| | | ACCAACTCGAGTTGGTGCTCTTCATC |
| | | TTGTTGA |

After annealing, the synthesized shRNA primer sequences were ligated into lentiviral shRNA expression vector pLKO.1 (purchased from Addgene). After mixed at a ratio of pLKO.1:dR8.9:VSVG=5:4:3, HEK 293T cells were transfected with the mixture using calcium phosphate method. Viruses were collected 48 h after transfection. After low-speed centrifugation and filtration, virus suspension was used directly to infect target cells.

4. Reagents and Isolation of Nucleus and Chromatin Subfractions:

PIM1 inhibitor (4-[3-(4-chlorophenyl)-2,1-benzisoxazol-5-yl]-2-pyrimidine amine) was purchased from Tocris.
IL-4 and IL-6 were purchased from Apotech.
MG132 was purchased from Merck.
Protein AG-beads were purchased from Shanghai Yuekebio.

Isolation of cellular components: cells were lysed using cytoplasm extraction solution (10 mM Hepes, pH 7.9, 10 mM KCl, 0.1 mM EDTA, 1 mM DTT, 0.5 mM PMSF, 1′ complete protease inhibitor cocktail (Cat No 1-697-498; Roche Biochem), 1 mM Na₃VO₄), placed on ice for 15 minutes, then added to a final concentration of 0.6% NP-40. After lysis by vortex for 30 seconds, centrifuged at 12000 g for 30 seconds. The obtained supernatant is cytoplasmic fraction and precipitate is nucleus component. After nucleus component was dissolved using nuclear suspension (20 mM Hepes pH 7.9, 400 mM NaCl, 1 mM EDTA, 1 mM DTT, 1 mM PMSF, 1′ protease inhibitor cocktail reagent, 1 mM Na₃VO₄), rotated slowly and uniformly at 4° C. for 30 minutes, followed by centrifugation at 16,060 g at 4° C. for 15 min. The obtained insoluble precipitate is chromatin subtraction.

5. Immunoprecipitation:

Cells were lysed using RIPA buffer (20 mM Tris/HCL pH 7.5, 150 mM NaCl, 1% NP-40, 0.5% Na-DOC, 1 mM EDTA and 1 mM PMSF protease inhibitor, 1×Cocktail, 1 mM Na3VO4 phosphatase inhibitor, 1 mM NaF). Primary antibody was first added to cell lysate and incubated for 1 h, and then agarose beads coupled with Protein A/G were added for 1 h. After lysis and washing three times with RIPA buffer, bound proteins were detected using SDS-PAGE.

6. Immunoblot:

After protein samples were subjected to SDS-PAGE, transferred to nitrocellulose membrane, sealed in TBST with 5% skim milk for 1 h, primary antibody was added and incubated for 1 h, then HRP-conjugated secondary antibody was added and incubated for 1 h, and results shown by exposure with ECL substrates.

7. Gene Site-Directed Mutagenesis:

FOXP3 S422A mutant and PIM1 K67M mutant were obtained by PCR mutation (see Reference Document 5)

| | SEQ ID | |
|---|---|---|
| Primer | NO: | Sequence |
| FOXP3 S422A mutant | 7 | GAGCCAGAGGCCCGCCAGGTGTTCC |
| primer 1 | | AAC |
| FOXP3 S422A mutant | 8 | GTTGGAACACCTGGCGGGCCTCTGG |
| primer 2 | | CTC |
| PIM1 K67M mutant | 9 | GCCGGTGGCCATCATGCACGTGGAG |
| primer 1 | | AAGGAC |
| PIM1 K67M mutant | 10 | GTCCTTCTCCACGTGCATGATGGCC |
| primer 2 | | ACCGGC |

8. Protein Expression and Purification:

PIM1 gene and PIM1K67M gene were inserted into PET28a prokaryotic expression vector to obtain PET28a-PIM1 and PET28a-PIM1K67M. Then, BL21 competent cells were transformed to induce expression with IPTG, cells collected, lysed, and purified using nickel column (Qiagen), and finally purified proteins were obtained by fast protein liquid chromatography (GE). MBP-S422FOXP3 and MBP-FOXP3 proteins expression was expressed as described above, cells collected, lysed, and purified using amylose resin (NEB), proteins were eluted and dialyzed using standard buffer (25 mM Tris-HCl (pH 7.5), 150 mM NaCl, 10 mM β-mercaptoethanol, and 10% glycerol). Finally, all protein concentrations were measured using Brandford methods (Beyotime Institute of Technology).

9. In Vitro Phosphorylation Experiments:

1 μg 6His-PIM1 or 6His-PIM1K67M and 1 μg MBP-FOXP3 (total four groups, respectively: 6His-PIM1; MBP-FOXP3; MBP-FOXP3+6His-PIM1; MBP-FOXP3+6His-PIM1K67M) was added to 100 μl reaction solution containing 20 mM MOPS buffer (pH 7.4), 150 mM NaCl, 12.5 mM magnesium chloride, 1 mM manganese chloride, 1 mM DTT, 10 μM ATP. Evenly mixed, after incubation at 30° C. for 2 h, SDS sample buffer was added to terminate the reaction. Immnuoblot was performed on the above reaction systems. FOXP3 S422 phosphorylation in each system was analyzed using anti-pS422-FOXP3 antibody.

10. Cell Proliferation Detection Experiments:

Regulatory T cells were obtained from human peripheral blood mononuclear cells (purchased from Shanghai Blood Center) by antibody labeling and magnetic bead sorting. As mentioned above, after labeling effector T cells with 1 μM CFSE (Invitrogen), regulatory T cells and effector T cells were cultured for 80 h. Flow cytometry was used to detect fluorescence intensity changes in effector T cells, their proliferation status was analyzed.

### Example I-1

### Effect of Proto-Oncogene Encoded Protein PIM1 on FOXP3

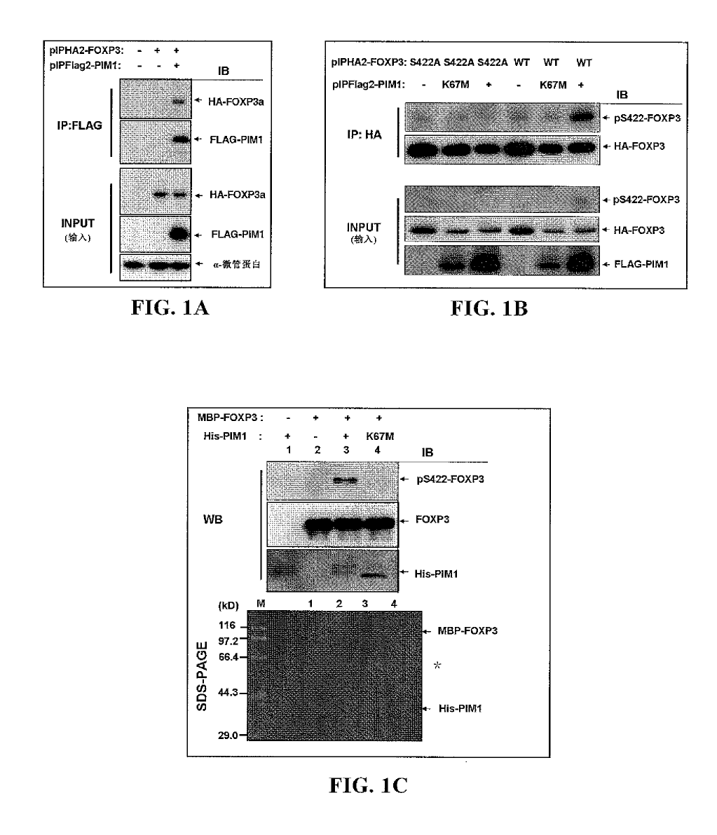

HEK293T cells were co-transfected with HA-FOXP3 and FLAG-PIM1. After 48 h, cells were collected. After lysed with RIPA buffer, immunoprecipitated using FLAG antibody, and then immuoblot was performed using corresponding antibodies (results shown in FIG. 1A). The results show: FLAG-PIM1 can be co-precipitated with HA-FOXP3.

HEK 293T cells were co-transfected with HA-FOXP3 or its Ser422 mutant and FLAG-PIM1 or its enzymatic activity-deficient mutant K67M. After 48 h, cells were collected. After lysis, immunoprecipitated using HA antibody and then immunoblot analysis was performed using pS422-FOXP3 antibody and other suitable antibodies (results shown in FIG. 1B). The results show: FLAG-PIM1 can phosphorylate Ser422 of HA-FOXP3.

Immunoblot analysis was performed after MBP-FOXP3 and His-PIM1 and its K67M mutants were expressed by prokaryotic cells and FPLC purified, and reacted in vitro, in the presence of ATP. SDS-PAGE was performed on the total proteins in the reaction system. After protein gel was stained by Lumitein™, and visualized under UV light (results shown in FIG. 1C). The results show: FOXP3 Ser422 phosphorylation can only be detected in MBP-FOXP3 and His-PIM1 in vitro phosphorylation experimental system.

The above results show: the proto-oncogene encoded protein PIM1 can bind FOXP3 and specifically catalyzes FOXP3 Ser422 phosphorylation. In other words, FOXP3 binds PIM1, and is phosphorylated at Ser422 by PIM1.

### Example I-2

### Inflammatory Cytokines IL-6 and IL-4 can Induce PIM1 Expression

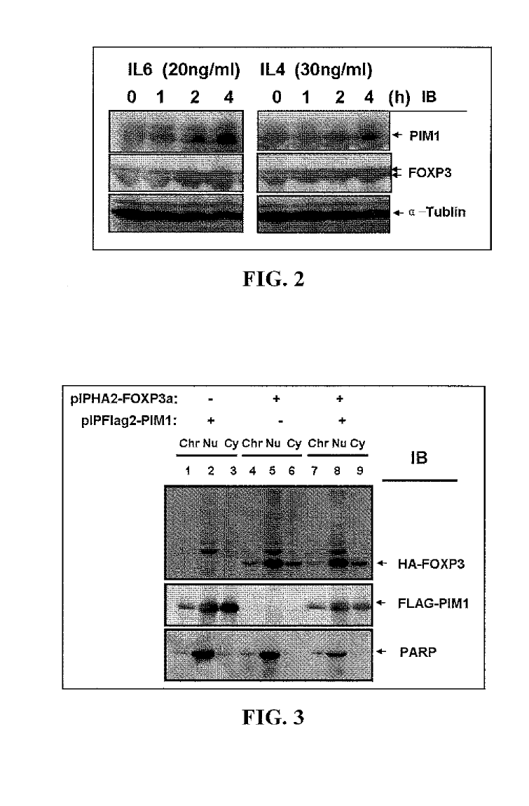

SZ4.03 cells were treated with IL-6 (20 ng/ml) or IL-4 (30 ng/ml), respectively, for 0 h, 1 h, 2 h, and 4 h. Cells were collected and lysed by boiling directly in SDS sample buffer, and then samples were analyzed by immunoblot (primary antibodies are PIM1 antibody and FOXP3 antibody) (results shown in FIG. 2).

The results show: after IL-6 or IL-4 treatment, in SZ4.03 lymphocyte cell line that highly expresses FOXP3, PIM1 expression gradually increases as treatment time increases; whereas there is no significant difference in FOXP3 expression levels.

The results show: two kinds of inflammatory cytokines, IL-4 and IL-6, can stimulate endogenous PIM1 expression.

### Example I-3

### PIM1 Inhibits FOXP3 Binding to Chromatin

HA-FOXP3 and FLAG-PIM1 were co-transfected into HEK 293T cells. After 48 h, cells were collected and chromatin (Chr), cytoplasmic (Cy) and nucleus (Nu) were isolated. Immunoblot analysis was performed on the isolated samples (results shown in FIG. 3).

The results show: when FOXP3 and PIM1 are present together, as compared with that when only one of them is present, the amount of FOXP3 binding to chromatin is significantly reduced; whereas the amount of FOXP3 in nucleus and cytoplasm remains unchanged.

The results show: phosphorylation modification exists as one of FOXP3 post-translational modifications. PIM1 can inhibit FOXP3 binding to chromatin, thereby inhibiting the transcription of FOXP3 target genes.

### Example I-4

### PIM1 Negatively Regulates FOXP3 Transcription Regulatory Activity

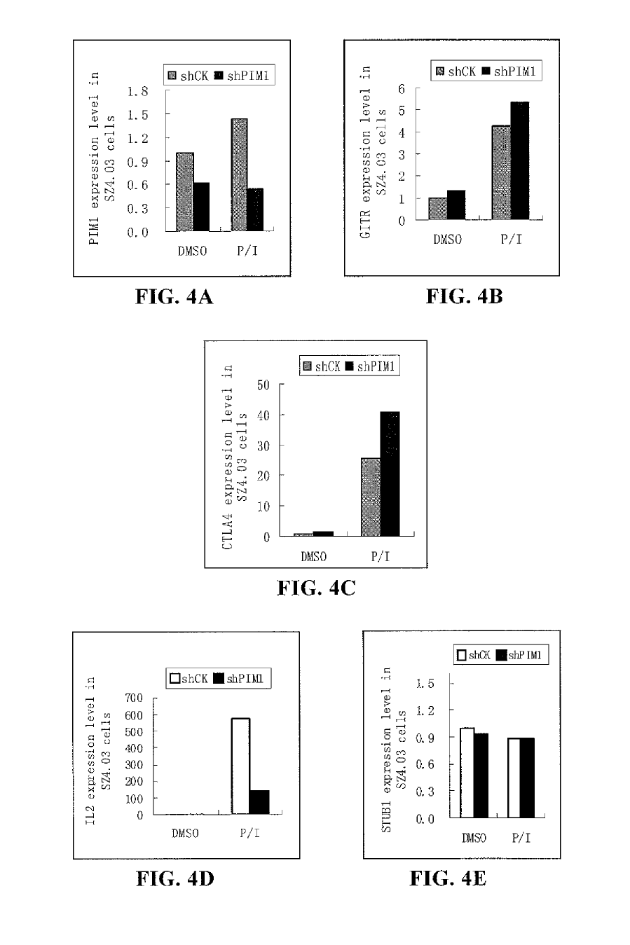

SZ4.03 cells that highly expresses FOXP3 were infected with viral vectors carrying the negative control shRNA (shCK) and PIM1 shRNA (shPIM1) packaged using lentiviral packaging system, followed by puromycin (50 ng/ml) selection and DMSO or PMA/inomycin (P/I) for 4 h. Total RNA was extracted and reverse transcribed to cDNA. Then, the mRNA expression levels of PIM1 (results shown in FIG. 4A), the mRNA expression levels of FOXP3-positively regulated target genes (GITR and CTLA4) (results shown in FIGS. 4B and 4C), the mRNA expression levels of FOXP3-negatively regulated target gene (IL-2) (results shown in FIG. 4D), the mRNA expression levels of non-FOXP3 target gene (STUB1) (results shown in FIG. 4E) were analyzed by quantitative PCR.

The results show: as compared with the control shCK, shPIM1 can significantly reduce the mRNA expression levels of PIM1. shPIM1 enhances the mRNA expression levels of FOXP3-positively regulated target genes (GITR and CTLA4), reduces the mRNA expression levels of FOXP3-negatively regulated target gene (IL-2), and does not affect the mRNA expression levels of non-FOXP3 target gene (STUB1).

The results show: PIM1 can negatively regulate FOXP3 transcription regulatory activity; shPIM1 can positively regulate FOXP3 transcription regulatory activity, thereby affect the mRNA expression levels of FOXP3 target genes.

### Example I-5

### PIM1 Inhibitors can Increase CLTA4+ FOXP3+ Treg and CD25+ FOXP3+ Treg Cell Levels

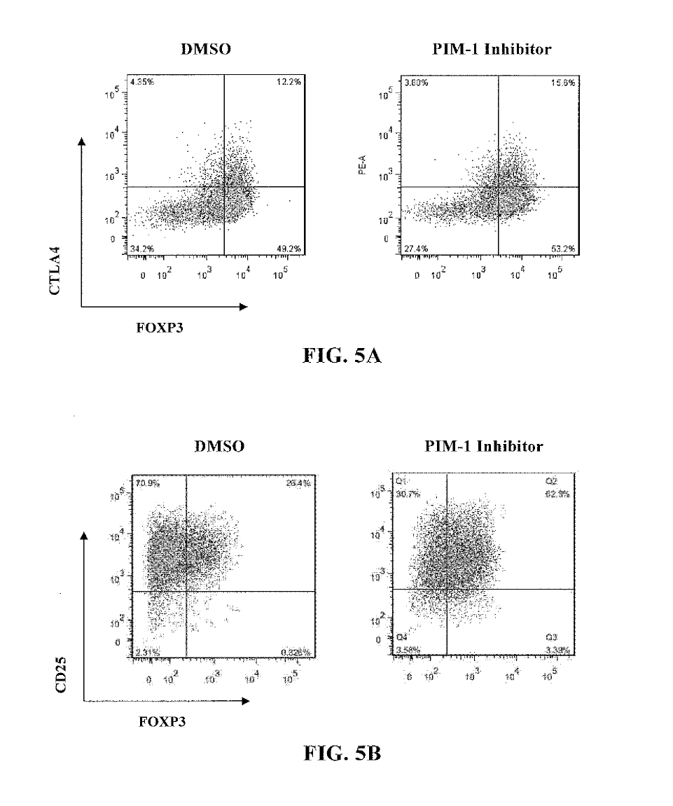

After human CD4+ CD25+ CD127^{low} natural Treg (nTreg) were sorted by BD FACS Aria II flow sorter, and expanded through Bead-CD3/CD28 and IL-2 (500 U/ml). DMSO or PIM1 inhibitor (4-[3-(4-chlorophenyl)-2,1-benzisoxazol-5-yl]-2-pyrimidinamine, 100 nM) was added and treated for 24 h, and the cells were stained with APC-FOXP3 and PE-CD25 antibody for flow cytometry analysis (results shown in FIG. 5A). The expanded nTreg were rested for 24 h. DMSO or PIM1 inhibitor (100 nM) was then added and treated for 48 h, followed by staining with APC-FOXP3 and PE-CD25 antibody for flow cytometry analysis (results shown in FIG. 5B).

The results show: as compared with DMSO control group, PIM1 inhibitor can significantly increase CLTA4+ FOXP3+ Treg and CD25+ FOXP3+ Treg cell levels.

The results show: PIM1 inhibitor can positively regulate FOXP3 expression levels and increase CLTA4+ FOXP3+ Treg and CD25+ FOXP3+ Treg cell levels.

### Example I-6

### PIM1 Inhibitors can Enhance Immunosuppressive Activity of nTreg

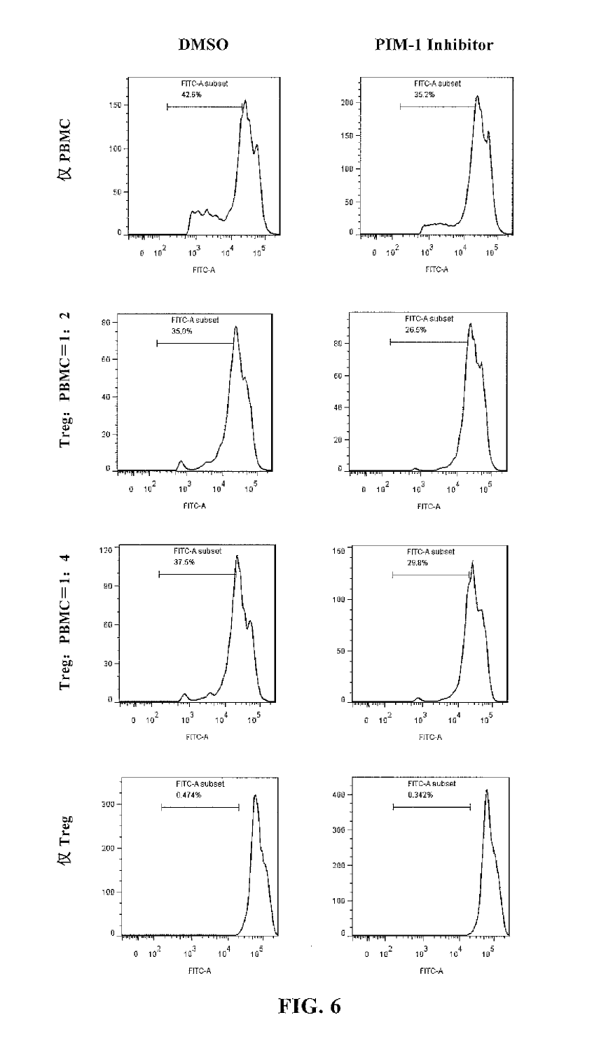

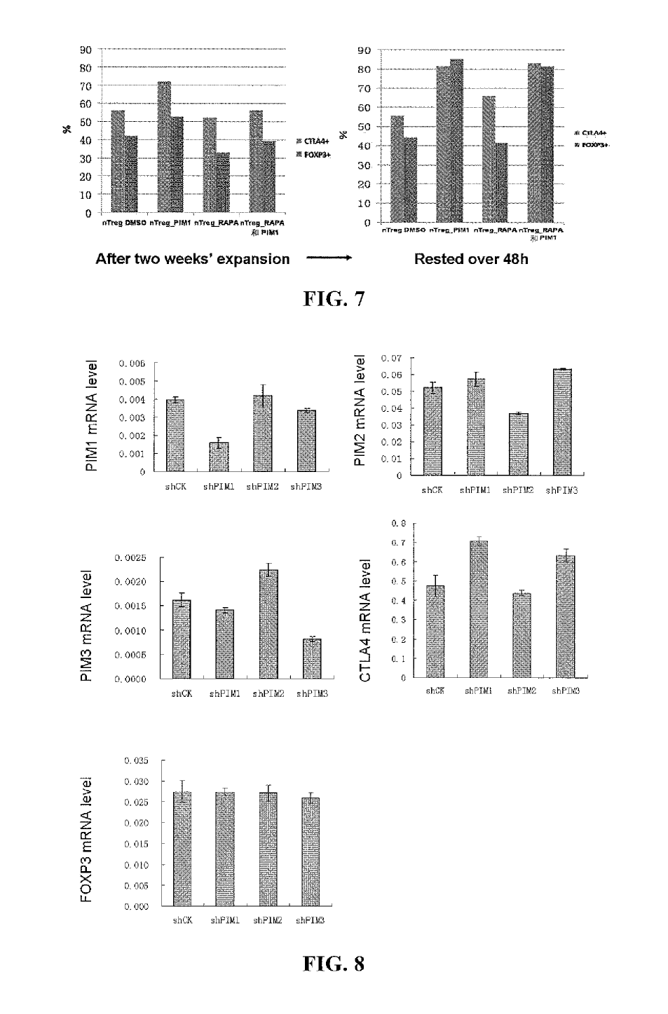

The expanded nTreg were mixed with CFSE-labeled human PBMC (2×10⁵ cells/well) at the corresponding ratio (i.e., PBMC only, Treg:PBMC=1:2 or 1:4, or Treg only) in 96-well U-shaped well. 5000 Bead-CD3/CD28 were added to each well. Two groups of cells were treated, respectively, with DMSO and PIM1 inhibitors, cultured for 5 days, followed by detection of cell proliferation levels (results shown in FIG. 6).

The results show: as compared with DMSO-treated group, PBMC proliferation was inhibited more in PIM1 inhibitor-treated group.

The results show: PIM1 inhibitors can enhance immunosuppressive activity of nTreg.

### Example I-7

### PIM1 Inhibitors Promote FOXP3+ and CTLA4+ nTreg Cell Expansion

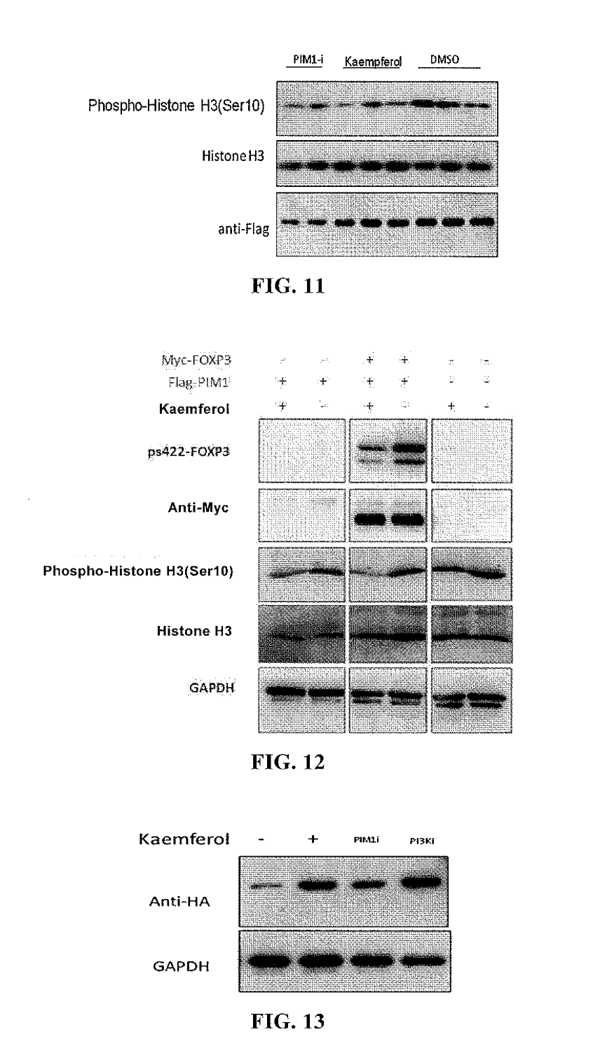

500 U/ml of IL-2 was added to 2×10⁵ nTreg cells. nTreg cells were expanded at a 3:1 ratio of CD3/CD28 T cell dynabeads and cells. After two weeks, part of the expanded nTreg cells were collected for FACS detection. Dynabeads were removed from the remaining cells and rested for 48 h. Cells were collected again for FACS detection of FOXP3 and CTLA4. Working concentrations of Rapamycin and PIM1 inhibitors are both 100 nM (results shown in FIG. 11).

The results show: comparing the PIM1 inhibitor-treated experimental group with the control group, PIM1 inhibitors can promote FOXP3+ and CTLA4+ nTreg cell expansion.

### Example I-8

### PIM1 Gene Knockout can Enhance FOXP3 Transcription Regulatory Activity

500 U/ml of IL-2 was added to 2×10⁵ nTreg cells. Primary nTreg cells were activated at a 3:1 ratio of CD3/CD28 T cell dynabeads and cells for 4 h, followed by infection of lentiviruses encoding PIM1, PIM2, and PIM3 shRNA overnight. Cells were cultured for 1 week without removing dynabeads, and then the mRNAs level of FOXP3 and its target gene CTLA4 were detected (results shown in FIG. 12).

The results show: the mRNA levels of CTLA4 increase in shPIM1 knockout nTreg cells, indicating that PIM1 gene can regulate FOXP3 transcription activity.

### Example II

The objective of this part of embodiments is to select small molecules capable of specifically affecting FOXP3+ Treg cell function and to develop agonists capable of affecting FOXP3 protein function; to detect the effect of these inhibitors on Treg cell function; and to provide new therapeutic approaches for the treatment of autoimmune diseases, organ transplantation, etc.

Shanghai Institute of Quality and Standardization provided Chinese herb medicine small molecule standard library. According to the retrieved information, there are 95 small molecules related to inflammation. Using molecular immunology-related technologies, we found, for the first time, that kaempferol affects FOXP3 post-translational modification and FOXP3 stability.

Relevant information about Kaempferol:

### Experiment Code: IPS-001

### Molecular Formula: C₁₅H₁₀O₆

### Molecular Weight: 286.24

### Structure:

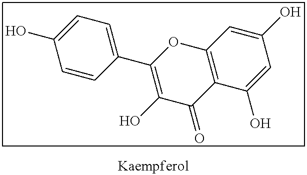 [SMILES: O=c1c(O)c(-c2ccc(O)cc2)oc2cc(O)cc(O)c12]

Materials and Methods

1. Plasmids and Vectors:

N-terminal Myc-FOXP3, Flag-PIM1, Flag-PIM2, and Flag-PIM3 were constructed and cloned according to Reference 4.
PIM1, PIM2, and PIM3 genes were obtained by amplification from human peripheral blood mononuclear cell cDNA library.

Primers were designed according to the sequences downloaded from Genbank as follows:

| | SEQ ID | |
|---|---|---|
| Primer | NO: | Sequence |
| PIM1-upstream | 1 | 5′-GAGGAATTCGATGCTCTTGTCCAAAAT |
| primer | | CAACTC-3′ |
| PIM1- | 2 | 5′-GAGGGGAGGACCTGCCAGAAAG-3′ |
| downstream | | |
| primer | | |
| PIM2-upstream | 11 | 5′-CACGAATTCTCCATGTTGACCAAGCCT |
| primer | | CTACAG-3′ |
| PIM2- | 12 | 5′-GAGGAAGGCTCCTTTGTAGGATTG-3′ |
| downstream | | |
| primer | | |
| PIM3-upstream | 13 | 5′-GTAGAATTCATGCTGCTCTCCAAGTTC |
| primer | | GGCTC-3′ |
| PIM3- | 14 | 5′-CTAGCTCCCAGTCAGGTGCAGCTC-3′ |
| downstream | | |
| primer | | |

Cell transfection: Lipofection was performed using Lipofectamin 2000 (Invitrogen) (performed according to operating instructions). Samples were collected at 48 h after transfection for analysis.

Human embryonic kidney cell line HEK293 was co-transfected with FUGW-TAP-FOXP3, dR8.9, and VSV-G. Cell culture supernatants were collected at 48 and 72 h after transfection.

shPIM1 primer sequences were designed as follows:

| | SEQ ID | |
|---|---|---|
| Primer | NO: | Sequence |
| ShPIM1 primer 1 | 3 | CCGGGATACTCTCTTCTTCTCATAG |
| | | CTCGAGCTATGAGAAGAAGAGAGTA |
| | | TCTTTTT |
| ShPIM1 primer 2 | 4 | AATTAAAAAGATACTCTCTTCTTCT |
| | | CATAGCTCGAGCTATGAGAAGAAGA |
| | | GAGTATC |
| ShPIM2 primer 1 | 15 | CCGGGCCCAGGATCTCTTTGACTAT |
| | | CTCGAGATAGTCAAAGAGATCCTGG |
| | | GCTTTTT |
| ShPIM2 primer 2 | 16 | AATTAAAAAGCCCAGGATCTCTTTG |
| | | ACTATCTCGAGATAGTCAAAGAGAT |
| | | CCTGGGC |
| ShPIM3 primer 1 | 17 | CCGGCGCCTGTCAGAAGATGAACAT |
| | | CTCGAGATGTTCATCTTCTGACAGG |
| | | CGTTTTT |
| ShPIM3 primer 2 | 18 | AATTAAAAACGCCTGTCAGAAGATG |
| | | AACATCTCGAGATGTTCATCTTCTG |
| | | ACAGGCG |

shCK primer sequences are as follows:

| | SEQ ID | |
|---|---|---|
| Primer | NO: | Sequence |
| ShCK primer 1 | 5 | CCGGTCAACAAGATGAAGAGCACCAACTCG |
| | | AGTTGGTGCTCTTCATCTTGTTGTTTTTG |
| ShCK primer 2 | 6 | AATTCAAAAACAACAAGATGAAGAGCACCA |
| | | ACTCGAGTTGGTGCTCTTCATCTTGTTGA |

After annealing, the synthesized snRNA primer sequences were ligated into shRNA lentiviral expression vector pLK0.1 (purchased from Addgene). After mixed at a ratio of pLK0.1:dR8.9:VSVG=5:4:3, they were transfected into HEK 293T cells by calcium phosphate method. Viruses were collected 48 h after transfection. Virus suspensions, after low-speed centrifugation and filtration, were used directly to infect target cells.

2. Antibodies:

Flag antibody (M2) was purchased from Sigma. FOXP3 antibody (hFOXY) was purchased from eBioscience. H3S10 (Phospho-Histone) and histone H3 antibodies were purchased from Cell signaling pS422-FOXP3 antibody was custom made by Abmart Inc. GAPDH antibody was purchased from Abmart Inc. β-tubulin was purchased from Tianjin Sungene Biotech Co., Ltd. HRP-conjugated anti-mouse secondary antibody was purchased from Promega.

3. Cells and Treatments:

Human HEK293T (purchased from the Chinese Academy of Sciences Cell Bank (catalog number: GNHu17)) was cultured in DMEM (Dulbecco's modified Eagle's medium) containing 10% FBS, 100 units/ml penicillin-streptomycin, at 37° C. under 5% CO₂.

Jurkat E6.1 T cells (purchased from the Chinese Academy of Sciences Cell Bank (catalog number: TCHU123)) were cultured in 1640 medium containing 10% FBS, 100 units/ml penicillin-streptomycin, 100 units/ml non-essential amino acids, and 100 units/ml sodium pyruvate, at 37° C. under 5% CO₂.

Cell transfection: Lipofection was performed using Lipofectamin 2000 (Invitrogen). Samples were collected 48 h after transfection for analysis.

Human embryonic kidney cell line HEK293 was co-transfected with FUGW-HA-FOXP3, del 8.9 and VSV-G. Cell culture supernatants were collected 48 h and 72 h after transfection. Viruses were purified by ultracentrifugation and used to infect Jurkat E6.1 to ultimately obtain Jurkat cell lines stably expressing HA-FOXP3.

4. Reagents and Isolation of Cellular Components:

PIM1 inhibitor (4-[3-(4-chlorophenyl)-2,1-benzisoxazol-5-yl]-2-pyrimidinamine) was purchased from Tocris. PI3K-inhibitor (LY294002) was purchased from Beyotime Institute of Biotechnology. PMA was purchased from Beyotime. LPS was purchased from Invivogen. Kaempferol was purchased from Shanghai Institute of Quality and Standardization.

Cells were lysed with cytoplasm extraction solution (10 mM Hepes, pH 7.9, 10 mM KCl, 0.1 mM EDTA, 1 mM DTT, 0.5 mM PMSF, 1′ complete protease inhibitor cocktails (Cat No 1-697-498; Roche Biochem), 1 mM Na₃VO₄), placed on ice 30 minutes, centrifuged at 12000 g for 30 seconds.

5. Immunoblot:

Protein samples were subjected to SDS-PAGE, and then transferred to nitrocellulose membrane, which was then sealed in TBST containing 5% nonfat dry milk or 3% bovine serum albumin for 1 h. Primary antibody was added and incubated for 1 h and then HRP-conjugated secondary antibody was added and incubated for 1 h, and results shown by exposure with ECL substrates.

6. Primary Cell Sorting and Cell Proliferation Assay Experiments:

Human peripheral blood mononuclear cells (purchased from Shanghai Blood Center) were simultaneously stained with CD4, CD25, and CD127 antibodies, and then the CD4+ CD25^{hi} CD127^{low} cell populations were selected by FACSAriaII cell sorter. The selected NTreg cells were cultured in vitro in X-VIVO (Lonza) medium containing 1% GlutaMax (GIBCO), 1% NaPyr (GIBCO), and 500 U IL2.

Expansion process requires the presence of CD3/CD28 T cell Dynabeads (Invitrogen, Catalog No.:111-41D). The ratio of Dynabeads and cells is 3:1. The phenotype of expanded nTreg cells was identified by detecting the expression levels of FOXP3. As mentioned above, after effector T cells were labeled with 1 μM CFSE (Invitrogen), regulatory T cells were cultured with effector T cells for 80 h. Fluorescence intensity changes in effector T cells were detected by flow cytometry, and analyzed for their proliferation status.

### Example II-1

### Kaempferol Inhibits FOXP3 S422 Phosphorylation in a Dose-Dependent Manner

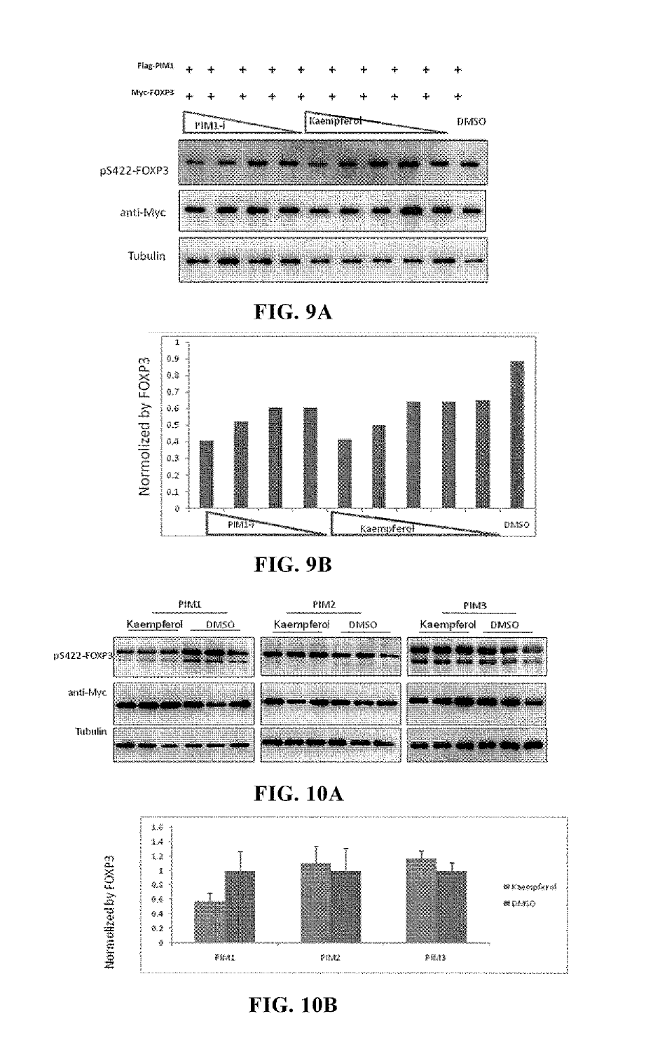

HEK293T cells were co-transfected with MYC-FOXP3 and FLAG-PIM1. 36 h later, cells were treated with 50 nM, 10 nM, 2 nM, and 0.4 nM of PIM1 inhibitor and 50 μM, 10 μM, 2 μM, 0.4 μM, and 0.08 μM of kaempferol for 12 h. After 48 h, cells were collected and lysed with cell lysis buffer. The levels of pS422-FOXP3, FOXP3, and β-tubulin were directly detected by immunoblot (results shown in FIG. 9A). FIG. 1A was analyzed using Image) software (results shown in FIG. 9B).

The results show: Kaempferol inhibits FOXP3 S422 phosphorylation in a dose-dependent manner.

### Example II-2

### Kaempferol can Specifically Inhibit FOXP3 S422 Phosphorylation Induced by PIM1

HEK293T cells were co-transfected, respectively, with Myc-FOXP3+ FLAG-PIM1, Myc-FOXP3+ FLAG-PIM2, and Myc-FOXP3+ FLAG-PIM3. 36 h later, cells were treated with 50 μM kaempferol for 12 h. After 48 h, cells were collected and lysed with cell lysis buffer. The levels of pS422-FOXP3, FOXP3, and β-tubulin were directly detected by immunoblot (results shown in FIG. 10A). FIG. 10A was analyzed using ImageJ software (results shown in FIG. 10B).

The results show: kaempferol does not affect FOXP3 phosphorylation levels induced by PIM2 and PIM3, but specifically inhibits FOXP3 S422 phosphorylation induced by PIM1.

### Example II-3

### Kaempferol can Inhibit Histone H3 S10 Phosphorylation Induced by PIM1

HEK293T cells were co-transfected with MYC-FOXP3 and FLAG-PIM1. 36 h later, cells were treated with 50 μM of kaempferol for 12 h. Cells were collected at 48 h after transfection and directly sonicated in SDS-loading buffer, and lysed by boiling. The levels of (Phospho-Histone) H3S10, histone H3, and PIM1 were directly detected using immunoblot (results shown in FIG. 11).

This results show: PIM1 can be recruited by MYC proto-oncogene to its target genes and phosphorylates histone H3 at Ser10 to initiate target gene transcription. Namely, histone H3 S10 is one of PIM1 phosphorylation substrates. The experimental results show kaempferol does not affect PIM1 expression levels, but inhibits histone H3 S10 phosphorylation induced by PIM1.

### Example II-4

### Kaempferol Only Inhibits FOXP3 S422 Phosphorylation and Histone H3 S10 Phosphorylation Induced by PIM1

HEK293T cells were transfected with FLAG-PIM1, and co-transfected with MYC-FOXP3 and FLAG-PIM1. 36 h later, HEK293 was used as blank control group and each group was treated with 50 μM of kaempferol for 12 h, respectively. Cells were collected after 48 h and lysed with cell lysis buffer. The levels of pS422-FOXP3, FOXP3, histone H3 Ser10 phosphorylation, histone 113, and GAPDH were directly detected by immunoblot (results shown in FIG. 12).

The results show: kaempferol treatment can inhibit FOXP3 S422 phosphorylation and histone H3 S10 phosphorylation; while it does not affect histone H3 S10 phosphorylation in the control group.

The results show: kaempferol specifically inhibits histone H3 S10 phosphorylation levels induced by PIM1.

### Example II-5

### Kaempferol can Inhibit FOXP3 Protein Degradation Induced by PMA/LPS Stimulation

Jurkat cells stably expressing HA-FOXP3 were treated with PMA (25 ng/ml) and LPS (1 ng/ml). Each sample was added 50 μM kaempferol, 100 nM PIM1 inhibitor (i.e., 4-[3-(4-chlorophenyl)-2,1-benzisoxazol-5-yl]-2-pyrimidinamine), and 10 μg/ml PI3K inhibitor (LY294002). DMSO serves as a control. Cells were collected after 24 h. The levels of FOXP3 and GAPDH were directly detected by immunoblot (results shown in FIG. 13).

The results show that: PMA and LPS co-stimulation can cause FOXP3 protein degradation in Jurkat cells stably expressing HA-FOXP3. Kaempferol, PIM1 inhibitor, and PI3K inhibitor can inhibit FOXP3 protein degradation.

### Example II-6

### Kaempferol Enhances FOXP3 and CTLA4 Expression in the Induced Treg

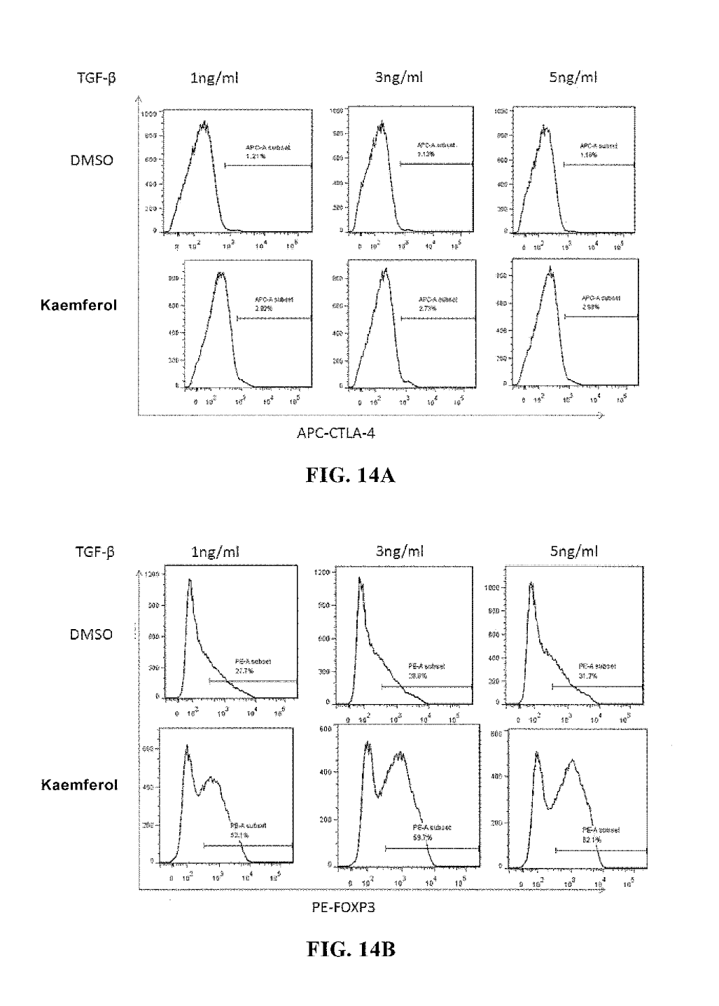

Human CD4+ CD45RA+ natural T cells (i.e., natural T cells rather than Treg cells) were sorted by BD FACS Aria II flow sorter and expanded using Bead-CD3/CD28 (2:1), IL-2 (100 U/ml), and TGF-β (1 ng/ml, 3 ng/ml, and 5 ng/ml) for 5 days to obtain the induced Treg cells. DMSO or kaempferol (50 uM) was added and treated for 48 h. After PE-FOXP3 and APC-CTLA4 antibody staining, flow cytometry analysis was performed (results as shown in FIGS. 14a and 14b).

The results show: as compared with the DMSO control group, kaempferol can significantly increase FOXP3 expression levels and CLTA4+ FOXP3+ Treg cell levels.

The results show: kaempferol can positively regulate FOXP3 expression levels and increase CLTA4+ FOXP3+ Treg cell levels.

All documents mentioned in the present invention are incorporated by reference in the present application, as if each reference was individually incorporated by reference. It should also be understood that, after reading the foregoing teachings of the present invention, those skilled in the art may make various modifications or changes of the present invention. These equivalents would similarly fall within the scope of the appended claims in the present application.

### REFERENCES

1. Belkaid, Y., Piccirillo, C. A., Mendez, S., Shevach, E. M., and Sacks, D. L. (2002). CD4+ CD25+ regulatory T cells control Leishmania major persistence and immunity. Nature 420, 502-507;
2. Belkaid, Y. and Rouse, B. T. (2005). Natural regulatory T cells in infectious disease. Nat Immunol 6, 353-360;
3. Li, B. and Greene, M. I. (2008). Special regulatory T-cell review: FOXP3 biochemistry in regulatory T cells—how diverse signals regulate suppression. Immunology 123, 17-19;
4. Li, B., Samanta, A., Song, X., Iacono, K. T., Bembas, K., Tao, R., Basu, S., Riley, J. L., Hancock, W. W., Shen, Y., et al. (2007). FOXP3 interactions with histone acetyltransferase and class II histone deacetylases are required for repression. Proc Natl Acad Sci USA 104, 4571-4576;
5. Qian, S., McDonough, H., Boellman, F., Cyr, D. M., Patterson, C., et al. (2006). CHIP-mediated stress recovery by sequential ubiquitination of substrates and Hsp70. Nature 440,551-555,
6. WO 2007/084775, Composition and method for modulation of suppressor T cell activation;
7. J. Todd Abrams, Stuart R. Lessin, Subrata K. Ghosh, Peter C. Nowell, William Ju., Eric C. Vonderheid, Alain H. Rook, Elaine Defreitas; Malignant and Nonmalignant T Cell Lines From Human T Cell Lymphotropic Virus Type I-negative Patients with Sezary Syndrome; The Journal of Immunology, Vol 146(5):1455-1462 (Mar. 1, 1991);
8. Miduturu C V, Deng X, Kwiatkowski N, Yang W, Brault L, Filippakopoulos P, Chung E, Yang Q, Schwaller J, Knapp 5, King R W, Lee J D, Herrgard S, Zarrinkar P, Gray N S; High-throughput kinase profiling: a more efficient approach toward the discovery of new kinase inhibitors. Chem. Biol. 2011 Jul. 29; 18(7):868-79. doi: 10.1016/j.chembiol. 2011.05.010;
9. Isaac M, Siu A, Jongstra J; The oncogenic PIM kinase family regulates drug resistance through multiple mechanisms. Drug Resist Updat. August-October, 2011; 14(4-5):203-11. doi: 10.1016/j.drup.2011.04.002. available on line May 20, 2011;
10. Blanco-Aparicio C, Camero A; Pim kinases in cancer: Diagnostic, prognostic and treatment opportunities. Biochem Pharmacol. Oct. 5, 2012. pii: S0006-2952(12)00649-1. doi: 10.1016/j.bcp.2012.09.018. [Early electronic publication]
11. Fathi A T, Arowojolu O, Swinnen I, Sato T, Rajkhowa T, Small D, Marmsater F, Robinson J E, Gross S D, Martinson M, Allen S, Kallan N C, Levis M; A potential therapeutic target for FLT3-ITD AML: PIM1 kinase. Leuk Res. 2012 February; 36(2):224-31. doi: 10.1016/j.leukres.2011.07.011. available on line Jul. 29, 2011;
12. Oleg Fedorov, Michael Sundstrom, Brian Marsden, Stefan Knapp; Insights for the development of specific kinase inhibitors by targeted structural genomics. Drug Discovery Today, Vol. 12 (9-10), May, 2007, pages 365-372;
13. Ogawa N, Yuki H, Tanaka A; Insights from Pim1 structure for anti-cancer drug design. Expert Opin Drug Discov. December 2012; 7(12):1177-92. doi: 10.1517/17460441.2012.727394. available on line Sep. 25, 2012;
14. Merkel A L, Meggers E, Ocker M; PIM1 kinase as a target for cancer therapy. Expert Opin Investig Drugs. April 2012; 21(4):425-36. doi: 10.1517/13543784.2012.668527. available on line Mar. 4, 2012.

1. A method for the treatment and/or the prevention of a disease or a symptom related to dysfunction of regulatory T cell immunomodulation comprises
administering to a subject in need thereof a composition comprising regulatory T cell or a composition for the regulation of regulatory T cell activity,
wherein the composition is prepared by contacting a starting material with a phosphorylation pathway-related factor, an agonist or an antagonist thereof, and wherein the phosphorylation pathway-related factor is selected from: proto-oncogene protein PIM1 and the coding sequence thereof.

2. The method of claim 1, wherein the phosphorylation pathway-related factor, the agonist or the antagonist thereof regulates activity of a regulator of the regulatory T cell, wherein the regulator is selected from the group: FOXP3 (forkhead box P3), IL-2 (interleukin 2), GITR (glucocorticoid-induced TNFR-related protein), CTLA4 (cytotoxic T-lymphocyte-associated protein 4), and a combination thereof.

3. The method of claim 2, wherein the phosphorylation pathway-related factor or the agonist thereof negatively regulates the activity of FOXP3, GITR or CTLA4 and positively regulates IL-2 activity, the antagonist of the phosphorylation pathway-related factor positively regulates the activity FOXP3, GITR, or CTLA4 activity and negatively regulates IL-2 activity.

4. The method of claim 1, wherein the agonist is selected from: a signal factor and the coding sequence thereof that induces PIM1 expression; the antagonist is selected from: an anti-PIM1.

5. The method of claim 4, wherein the agonist is selected from: IL-4 (interleukin 4), IL-6 (interleukin 6), TCR (T-cell receptor), and the coding sequence thereof.

6. The method of claim 1, wherein the antagonist is kaempferol or a pharmaceutically acceptable salt thereof.

7. The method of claim 1, wherein the starting material comprising a CD4+ T cell.

8. (canceled)

9. The method of claim 1, wherein the disease or the symptom related to dysfunction of regulatory T cell immunomodulation is a disease or a symptom related to the regulatory T cell having an abnormal immunosuppressive function.

10. The method of claim 1, wherein the composition is a pharmaceutical composition, a healthcare composition, a vaccine composition, or an adjuvant composition.

11. A method for the preparation of a composition comprising regulatory T cell, wherein the method comprises:
(a) providing a starting material comprising CD4+ T cell;
(b) contacting the starting material with a phosphorylation pathway-related factor, an agonist or an antagonist thereof to obtain the regulatory T cell, or with a composition comprising a regulatory T cell, wherein the phosphorylation pathway-related factor is selected from: proto-oncogene protein PIM1 and the coding sequence thereof.

12. The method of claim 11, wherein the method further comprises one or more steps selected from the group consisting of:
step (a′) before step (b), purifying or isolating the CD4+ T cell; and
step (c) after step (b), purifying or isolating the regulatory T cell or cells in the composition comprising the regulatory T cell.

13. A method of regulating regulatory T cell activity, wherein the method comprising:
contacting a sample or an object comprising a CD4+ T cell with a phosphorylation pathway-related factor, an agonist or an antagonist thereof, or with the composition prepared by the method of claim 11.

14. A reagent or a kit for the preparation of a composition comprising regulatory T cell or a composition for the regulation of regulatory T cell activity, comprising:
(a) one or more phosphorylation pathway-related factors selected from the group: proto-oncogene protein PIM1 and the coding sequence thereof; an agonist and an antagonist of the phosphorylation pathway-related factors; and
(b) a pharmaceutically, immunologically, or healthcare science acceptable carrier.

15. The reagent or the kit of claim 14, wherein the regulation is achieved by regulating the activity of a regulatory factor of the regulatory T cell, wherein the regulator is selected from the group: FOXP3, IL-2, GITR, CTLA4, and a combination thereof.

16. The reagent or the kit of claim 14, wherein the antagonist is kaempferol or a pharmaceutically acceptable salt thereof.

17. A method of selecting a substance that regulates regulatory T cell activity, wherein the method comprising
detecting FOXP3 protein stability or PIM1 activity, wherein the substance that regulates the regulatory T cell activity is selected by detecting FOXP3 phosphorylation induced by PIM1 or the FOXP3 protein stability.

18. The method of claim 7, wherein the CD4+ T cell is a CD4+ CD45RA+ natural T cell.

19. A method of regulating regulatory T cell activity, wherein the method comprising:
contacting a sample or an object comprising a CD4+ T cell with a phosphorylation pathway-related factor, an agonist or an antagonist thereof, or with the composition prepared by the method of claim 12.

20. The method of claim 4, wherein the chemical inhibitor of PIM1 is selected from 4-[3-(4-chlorophenyl)-2,1-benzisoxazol-5-yl]-2-pyrimidinamine, 3-cyano-4-phenyl-6-(3-bromo-6-hydroxy)phenyl-2(1H)-pyridinone, 2-hydroxy-3-cyano-4-phenyl-6-(3-bromo-6-hydroxyphenyl)pyridine, and a racemic mixture of a pyridocarbazole-cyclopentadienyl Ruthenium complex


SEQUENCE LISTING (18 sequences)

SEQ ID NO: 1 - DNA, 33 bp, Artificial Sequence
  Primer
gaggaattcgatgctcttgtccaaaatcaactc

SEQ ID NO: 2 - DNA, 22 bp, Artificial Sequence
  Primer
gaggggaggacctgccagaaag

SEQ ID NO: 3 - DNA, 57 bp, Artificial Sequence
  Primer
ccgggatactctcttcttctcatagctcgagctatgagaagaagagagtatcttttt

SEQ ID NO: 4 - DNA, 57 bp, Artificial Sequence
  Primer
aattaaaaagatactctcttcttctcatagctcgagctatgagaagaagagagtatc

SEQ ID NO: 5 - DNA, 59 bp, Artificial Sequence
  Primer
ccggtcaacaagatgaagagcaccaactcgagttggtgctcttcatcttgttgtttttg

SEQ ID NO: 6 - DNA, 59 bp, Artificial Sequence
  Primer
aattcaaaaacaacaagatgaagagcaccaactcgagttggtgctcttcatcttgttga

SEQ ID NO: 7 - DNA, 28 bp, Artificial Sequence
  Primer
gagccagaggcccgccaggtgttccaac

SEQ ID NO: 8 - DNA, 28 bp, Artificial Sequence
  Primer
gttggaacacctggcgggcctctggctc

SEQ ID NO: 9 - DNA, 31 bp, Artificial Sequence
  Primer
gccggtggccatcatgcacgtggagaaggac

SEQ ID NO: 10 - DNA, 31 bp, Artificial Sequence
  Primer
gtccttctccacgtgcatgatggccaccggc

SEQ ID NO: 11 - DNA, 33 bp, Artificial Sequence
  Primer
cacgaattctccatgttgaccaagcctctacag

SEQ ID NO: 12 - DNA, 24 bp, Artificial Sequence
  Primer
gaggaaggctcctttgtaggattg

SEQ ID NO: 13 - DNA, 32 bp, Artificial Sequence
  Primer
gtagaattcatgctgctctccaagttcggctc

SEQ ID NO: 14 - DNA, 24 bp, Artificial Sequence
  Primer
ctagctcccagtcaggtgcagctc

SEQ ID NO: 15 - DNA, 57 bp, Artificial Sequence
  Primer
ccgggcccaggatctctttgactatctcgagatagtcaaagagatcctgggcttttt

SEQ ID NO: 16 - DNA, 57 bp, Artificial Sequence
  Primer
aattaaaaagcccaggatctctttgactatctcgagatagtcaaagagatcctgggc

SEQ ID NO: 17 - DNA, 57 bp, Artificial Sequence
  Primer
ccggcgcctgtcagaagatgaacatctcgagatgttcatcttctgacaggcgttttt

SEQ ID NO: 18 - DNA, 57 bp, Artificial Sequence
  Primer
aattaaaaacgcctgtcagaagatgaacatctcgagatgttcatcttctgacaggcg